(19) United States
(12) Patent Application Publication
(10) Pub. No.: US 2024/0010746 A1
(43) Pub. Date: Jan. 11, 2024

(54) TREATMENT METHODS USING ANTI-CD73 AND ANTI-PD-L1 ANTIBODIES AND CHEMOTHERAPY

(71) Applicant: MEDIMMUNE, LLC, GAITHERSBURG, MD (US)

(72) Inventors: Rakesh KUMAR, GAITHERSBURG, MD (US); Judson ENGLERT, GAITHERSBURG, MD (US); Zachary COOPER, GAITHERSBURG, MD (US); Philip Lloyd Martin, GAITHERSBURG, MD (US)

(21) Appl. No.: 18/246,206

(22) PCT Filed: Sep. 22, 2021
(86) PCT No.: PCT/IB2021/058662

§ 371 Date: Mar. 22, 2023

Related U.S. Application Data
(60) Provisional application No. 63/082,162, filed on Sep. 23, 2020.
(60) Provisional application No. 63/084,900, filed on Sep. 29, 2020.

(51) Int. Cl. C07K 16/28; A61K 31/7068; A61K 31/337; A61K 31/555; A61K 31/519; A61K 31/513; A61P 35/00

(52) U.S. Cl. CPC C07K 16/2896; A61K 31/7068; A61K 31/337; A61K 31/555; A61K 31/519; A61K 31/513; C07K 16/2827; A61P 35/00

(57) ABSTRACT

This disclosure relates to methods of treating cancers in a human patient (e.g., metastatic pancreatic ductal adenocarcinoma (PD AC)), comprising administering an anti-CD73 antibody or antigen binding fragment thereof and chemotherapy. The disclosure also relates to methods for the treatment of tumors comprising administering an anti-CD73 antibody or antigen-binding fragment thereof in combination with a PD-L1 antibody or an antigen-binding fragment thereof and chemotherapy.

### 1. CROSS-REFERENCE TO RELATED APPLICATIONS

This International Application claims the priority benefit of U.S. Provisional Application No. 63/082,162, filed on Sep. 23, 2020 and U.S. Provisional Application No. 63/084,900, filed on Sep. 29, 2020, each of which is incorporated herein by reference in its entirety.

### 2. REFERENCE TO SEQUENCE LISTING SUBMITTED ELECTRONICALLY

The content of the electronically submitted sequence listing in ASCII text file (Name: CD73_211_PCT_Seglisting_ST25.txt; Size: 7,109 bytes; and Date of Creation: Sep. 20, 2021) filed with the application is incorporated herein by reference in its entirety.

### 3. FIELD

This disclosure relates to methods of using anti-CD73 and anti-PD-L1 antibodies and antigen binding fragments thereof for the treatment of cancers, e.g., metastatic pancreatic ductal adenocarcinoma (PDAC).

### 4. BACKGROUND

Pancreatic ductal adenocarcinoma (PDAC) is a malignancy with an extremely poor prognosis, as exemplified by a 1-year survival rate of approximately 18% for all stages of the disease and an estimated 5-year survival rate of less than 4% (Hidalgo M. et al., Pancreatology, 15(1):8-18n(2015)). In 2020, the number of estimated New Cases was 57,600, or 3.2% of all new cancer cases. Many PDAC patients have metastatic disease, and a large percentage have locally advanced disease not amenable to resection (Gillen S. et al., PLOS Medicine, 7(4):e1000267(2010)).

Immune-checkpoint inhibitors have shown significant antitumor activity and become the standard-of-care for multiple tumor types. Immunotherapy in combination with chemotherapy has shown the potential for additive or synergistic effects on clinical response across multiple tumor types.

CD73 or ecto-5′-nucleotidase (5′-NT) is ubiquitously expressed in a number of tissues. This protein is anchored to the cell membrane through a glycosylphosphatidylinositol (GPI) linkage, has ecto-enzyme activity, and plays a role in signal transduction. The primary function of CD73 is the conversion of extracellular nucleotides (e.g., 5′-AMP), to which cells are generally impermeable, to their corresponding nucleosides (e.g., adenosine), which can readily enter most cells. CD73 production of adenosine by the dephosphorylation of AMP, has been shown to regulate adenosine receptor engagement in many tissues, indicating that adenosine functions in cytoprotection, cell growth, angiogenesis and immunosuppression, and also plays a role in tumorigenesis.

CD73 expression on tumor cells has been reported in several types of cancer, including colorectal cancer, pancreatic cancer, bladder cancer, leukemia, lymphoma, glioma, glioblastoma, melanoma, ovarian cancer, thyroid cancer, esophageal cancer, prostate cancer, and breast cancer. Elevated CD73 expression has also been associated with tumor invasiveness, metastasis, and reduced patient survival time. CD73 generates an immunosuppressed environment, characterized by increased adenosine levels, which promote the development and progression of cancer. Notably, CD73 expression has been associated with a prometastatic phenotype in melanoma and breast cancer.

Programmed death-ligand 1 (PD-L1), also known as B7H1, is a 40 kDa transmembrane protein that is a major obstacle in anti-cancer immunity. PD-L1 binding to the programmed death receptor (PD-1), inactivates T-cells, protects tumor cells, and suppresses immune system detection, allowing for unchecked proliferation of cancer cells. PD-L1 also binds CD80, a co-stimulatory molecule. A wide range of tumorigenic and activated immune cell types naturally express PD-L1, including antigen presenting cells, macrophages, monocytes, B cells, T cells and non-hematopoietic cells. Further, inflammatory cytokines induce PD-L1 expression; including interferon gamma (IFNγ). Activated T-cells produced IFNγ, the most potent inducer of PD-L1. IFNγ in turn induces PD-L1 expression, promoting tumor protection, a mechanism known as adaptive immune resistance.

While immune-checkpoint inhibitors have shown great promise as cancer therapeutics, clinical benefits from immune-checkpoint inhibition have been modest. Accordingly, improved methods for reducing tumor-mediated immunosuppression are required.

### 5. SUMMARY

Provided herein are methods of treating a metastatic pancreatic ductal adenocarcinoma (PDAC) in a subject, the method comprising administering to the subject about 750 mg to about 3000 mg of an anti-CD73 antibody or antigen binding fragment thereof and chemotherapy, wherein a tumor sample obtained from the subject expresses CD73.

Also provided herein are methods of inhibiting the growth of a PDAC tumor in a subject, the method comprising administering to the subject about 750 mg to about 3000 mg of an anti-CD73 antibody or antigen binding fragment thereof and chemotherapy, wherein a tumor sample obtained from the subject expresses CD73. In some aspects, the anti-CD73 antibody or antigen binding fragment thereof comprises oleclumab or antigen-binding fragment thereof.

In some aspects, the oleclumab or antigen-binding fragment thereof is administered at a dose of 750 mg. In some aspects, the oleclumab or antigen-binding fragment thereof is administered at a dose of 1500 mg. In some aspects, the oleclumab or antigen-binding fragment thereof is administered at a dose of 2250 mg. In some aspects, the oleclumab or antigen-binding fragment thereof is administered at a dose of 3000 mg.

In some aspects, the dose of oleclumab or an antigen-binding fragment thereof is administered every two weeks for four doses and then every four weeks.

In some aspects, the chemotherapy comprises gemcitabine and nab-paclitaxel. In some aspects, the gemcitabine is administered at a dose of 1000 mg/m² and the nab-paclitaxel is administered at a dose of 125 mg/m². In some aspects, the chemotherapy is administered on days 1, 8, and 15 of a 28-day treatment cycle and then the cycle is repeated every 4 weeks.

In some aspects, the chemotherapy comprises mFOLFOX. In some aspects, the mFOLFOX comprises oxaliplatin administered at a dose of about 85 mg/m², leucovorin administered at a dose of about 400 mg/m², and 5-FU administered in a bolus of about 400 mg/m² followed by administration of a second dose of 5-FU at about 2400 mg/m². In some aspects, the chemotherapy is administered on days 1 and 15 of a 28-day treatment cycle and then the cycle is repeated every 4 weeks.

In some aspects of the methods provided herein, the oleclumab or antigen binding fragment thereof is administered at a dose of 750 mg every 2 weeks for four doses and then every 4 weeks in the treatment cycle, and wherein the chemotherapy comprises 1000 mg/m² gemcitabine and 125 mg/m² nab-paclitaxel and is administered on days 1, 8, and 15 of the 28-day treatment cycle and then the cycle is repeated every 4 weeks.

In some aspects, the oleclumab or antigen binding fragment thereof is administered at a dose of 1500 mg every 2 weeks for four doses and then every 4 weeks in the treatment cycle, and wherein the chemotherapy comprises 1000 mg/m² gemcitabine and 125 mg/m² nab-paclitaxel and is administered on days 1, 8, and 15 of the 28-day treatment cycle and then the cycle is repeated every 4 weeks.

In some aspects, the oleclumab or antigen binding fragment thereof is administered at a dose of 2250 mg every 2 weeks for four doses and then every 4 weeks in the treatment cycle, and wherein the chemotherapy comprises 1000 mg/m² gemcitabine and 125 mg/m² nab-paclitaxel and is administered on days 1, 8, and 15 of the 28-day treatment cycle and then the cycle is repeated every 4 weeks.

In some aspects, the oleclumab or antigen binding fragment thereof is administered at a dose of 3000 mg every 2 weeks for four doses and then every 4 weeks in the treatment cycle, and wherein the chemotherapy comprises 1000 mg/m² gemcitabine and 125 mg/m² nab-paclitaxel and is administered on days 1, 8, and 15 of the 28-day treatment cycle and then the cycle is repeated every 4 weeks.

In some aspects, of the methods provided herein, the oleclumab or antigen binding fragment thereof is administered at a dose of 750 mg every 2 weeks for four doses and then every 4 weeks in the treatment cycle, and wherein the chemotherapy comprises mFOLFOX and is administered on days 1 and 15 of the 28-day treatment cycle and then the cycle is repeated every 4 weeks.

In some aspects, the oleclumab or antigen binding fragment thereof is administered at a dose of 1500 mg every 2 weeks for four doses and then every 4 weeks in the treatment cycle, and wherein the chemotherapy comprises mFOLFOX and is administered on days 1 and 15 of the 28-day treatment cycle and then the cycle is repeated every 4 weeks.

In some aspects, the oleclumab or antigen binding fragment thereof is administered at a dose of 2250 mg every 2 weeks for four doses and then every 4 weeks in the treatment cycle, and wherein the chemotherapy comprises mFOLFOX and is administered on days 1 and 15 of the 28-day treatment cycle and then the cycle is repeated every 4 weeks.

In some aspects, the oleclumab or antigen binding fragment thereof is administered at a dose of 3000 mg every 2 weeks for four doses and then every 4 weeks in the treatment cycle, and wherein the wherein the chemotherapy comprises mFOLFOX and is administered on days 1 and 15 of the 28-day treatment cycle and then the cycle is repeated every 4 weeks.

In some aspects, the oleclumab or antigen binding fragment and the chemotherapy are administered simultaneously or sequentially.

In some aspects, the methods disclosed herein, further comprise administering to the subject about 1500 mg of an anti-PD-L1 antibody or antigen binding fragment thereof. In some aspects, the anti-PD-L1 antibody or antigen binding fragment thereof comprises durvalumab or antigen binding fragment thereof.

In some aspects, the durvalumab or antigen binding fragment thereof is administered at a dose of 1500 mg. In some aspects, the dose of durvalumab or antigen binding fragment thereof is administered every four weeks. In some aspects, the chemotherapy are administered simultaneously or sequentially.

In some aspects, the administration is parenteral. In some aspects, the administration is intravenous. In some aspects, the administration is via intravenous infusion.

In some aspects, the subject is human. In some aspects, the human subject is an adult ≥18 years of age with histologically or cytologically confirmed pancreatic adenocarcinoma. In some aspects, the subject has previously untreated first-line metastatic PDAC (1L metastatic PDAC). In some aspects, the subject has previously untreated second-line metastatic PDAC (2L metastatic PDAC).

In some aspects, the CD73 expression of a tumor sample obtained from the subject is evaluated by an immunohistochemistry (IHC) method. In some aspects, the IHC method is an automated IHC method.

In some aspects, a tumor sample obtained from the subject expresses high levels of CD73.

In some aspects, the IHC method comprises IHC scoring. In some aspects, the IHC scoring is defined by scoring the staining intensity of cells expressing CD73 within the tumor sample with the value 0, 1, 2, or 3. In some aspects, the IHC scoring is defined by scoring the staining intensity of cells expressing CD73 within the tumor sample with the value 1, 2, or 3.

In some aspects, the percentage of cells expressing CD73 at each value in the tumor sample is calculated. In some aspects, the tumor sample comprises cells having staining intensities of 1, 2, and 3. In some aspects, the tumor sample comprises at least about 50% to about 90% of cells having staining intensities of 1, 2, and 3. In some aspects, the tumor sample comprises at least about 50%, about 60%, about 70%, about 80%, or about 90% cells having staining intensities of 1, 2, and 3.

In some aspects, the tumor sample comprises cells having staining intensities of 2 and 3. In some aspects, the tumor sample comprises at least about 30% to about 70% of cells having staining intensities of 2 and 3. In some aspects, the tumor sample comprises at least about 30%, about 40%, about 50%, about 60%, or about 70% of cells having staining intensities of 2 and 3.

In some aspects of the methods disclosed herein, at least about 70% of the cells in a tumor sample obtained from the subject have a staining intensity score of at least 1.

In some aspects of the methods disclosed herein, at least about 50% of the cells in a tumor sample obtained from the subject have a staining intensity score of at least 2.

In some aspects of the methods disclosed herein, the anti-CD73 antibody or antigen binding fragment thereof comprises oleclumab which comprises a heavy chain variable domain and a light chain variable domain, wherein the heavy chain variable domain comprises CDR1, CDR2, and CDR3 sequences of SEQ ID NOs: 3-5, and wherein the light chain variable domain comprises CDR1, CDR2, and CDR3 sequences of SEQ ID NOs: 6-8.

In some aspects, wherein the oleclumab comprises a light chain variable domain comprising the amino acid sequence of SEQ ID NO: 1 and a heavy chain variable domain comprising the amino acid sequence of SEQ ID NO: 2.

In some aspects of the methods disclosed herein, the anti-PD-L1 antibody or antigen binding fragment thereof comprises durvalumab which comprises a heavy chain variable domain and a light chain variable domain, wherein the heavy chain variable domain comprises CDR1, CDR2, and CDR3 sequences of SEQ ID NOs: 11-13, and wherein the light chain variable domain comprises CDR1, CDR2, and CDR3 sequences of SEQ ID NOs: 14-16.

In some aspects, the durvalumab comprises a light chain variable domain comprising the amino acid sequence of SEQ ID NO: 9 and a heavy chain variable domain comprising the amino acid sequence of SEQ ID NO: 10.

In some aspects of the methods disclosed herein, the anti-CD73 antibody or antigen binding fragment thereof is administered every 2 weeks for four doses and then every 4 weeks in a treatment cycle, and wherein the chemotherapy is administered on days 1 and 15 of the 28-day cycle treatment cycle and then the cycle is repeated every 4 weeks.

In some aspects of the methods disclosed herein, the anti-CD73 antibody or antigen binding fragment thereof is administered every 2 weeks for four doses and then every 4 weeks in a treatment cycle, and wherein the anti-PD-L1 antibody or antigen binding fragment thereof is administered every four weeks in the treatment cycle, wherein the chemotherapy is administered on days 1, 8, and 15 of the 28-day treatment cycle, and then the cycle is repeated every 4 weeks.

Also provided herein are methods of inhibiting the growth of a tumor in a human subject comprising administering (i) oleclumab or antigen binding fragment thereof, (ii) durvalumab or antigen binding fragment thereof, and (iii) chemotherapy to the subject, wherein at least about 50% to about 90% of cells in a tumor sample obtained from the human subject have a staining intensity of 1, 2, or 3, as determined by IHC; and wherein the oleclumab or antigen binding fragment is administered to the subject at a dose of 750 mg every 2 weeks for four doses and then every 4 weeks in a treatment cycle; the durvalumab or antigen binding fragment thereof is administered to the subject at a dose of 1500 mg every four weeks in the treatment cycle; and the chemotherapy comprises 1000 mg/m² gemcitabine and 125 mg/m² nab-paclitaxel and is administered on days 1, 8, and 15 of the 28-day treatment cycle and then the cycle is repeated every 4 weeks.

In some aspects, the methods disclosed herein comprise administering (i) oleclumab or antigen binding fragment thereof, (ii) durvalumab or antigen binding fragment thereof, and (iii) chemotherapy to the subject, wherein at least about 50% to about 90% of cells in a tumor sample obtained from the human subject have a staining intensity of 1, 2, or 3, as determined by IHC; and wherein the oleclumab or antigen binding fragment is administered to the subject at a dose of 1500 mg every 2 weeks for four doses and then every 4 weeks in a treatment cycle; the durvalumab or antigen binding fragment thereof is administered to the subject at a dose of 1500 mg every four weeks in the treatment cycle; and the chemotherapy comprises 1000 mg/m² gemcitabine and 125 mg/m² nab-paclitaxel and is administered on days 1, 8, and 15 of the 28-day cycle and then the cycle is repeated every 4 weeks.

In some aspects, the methods disclosed herein comprise administering (i) oleclumab or antigen binding fragment thereof, (ii) durvalumab or antigen binding fragment thereof, and (iii) chemotherapy to the subject, wherein at least about 50% to about 90% of cells in a tumor sample obtained from the human subject have a staining intensity of 1, 2, or 3, as determined by IHC; and wherein the oleclumab or antigen binding fragment is administered to the subject at a dose of 2250 mg every 2 weeks for four doses and then every 4 weeks in a treatment cycle; the durvalumab or antigen binding fragment thereof is administered to the subject at a dose of 1500 mg every four weeks in the treatment cycle; and the chemotherapy comprises 1000 mg/m² gemcitabine and 125 mg/m² nab-paclitaxel and is administered on days 1, 8, and 15 of the 28-day cycle and then the cycle is repeated every 4 weeks.

In some aspects, the methods disclosed herein comprise administering (i) oleclumab or antigen binding fragment thereof, (ii) durvalumab or antigen binding fragment thereof, and (iii) chemotherapy to the subject, wherein at least about 50% to about 90% of cells in a tumor sample obtained from the human subject have a staining intensity of 1, 2, or 3, as determined by IHC; and wherein the oleclumab or antigen binding fragment is administered to the subject at a dose of 3000 mg every 2 weeks for four doses and then every 4 weeks in a treatment cycle; the durvalumab or antigen binding fragment thereof is administered to the subject at a dose of 1500 mg every four weeks in the treatment cycle; and the chemotherapy comprises 1000 mg/m² gemcitabine and 125 mg/m² nab-paclitaxel and is administered on days 1, 8, and 15 of the 28-day cycle and then the cycle is repeated every 4 weeks.

In some aspects, the methods disclosed herein comprise administering (i) oleclumab or antigen binding fragment thereof, (ii) durvalumab or antigen binding fragment thereof, and (iii) chemotherapy to the subject, wherein at least about 50% to about 90% of cells in a tumor sample obtained from the human subject have a staining intensity of 1, 2, or 3, as determined by IHC; and wherein the oleclumab or antigen binding fragment is administered to the subject at a dose of 750 mg every 2 weeks for four doses and then every 4 weeks in a treatment cycle; the durvalumab or antigen binding fragment thereof is administered to the subject at a dose of 1500 mg every four weeks in the treatment cycle; and the chemotherapy comprises mFOLFOX and is administered on days 1 and 15 of the 28-day treatment cycle and then the cycle is repeated every 4 weeks.

In some aspects, the methods disclosed herein comprise administering (i) oleclumab or antigen binding fragment thereof, (ii) durvalumab or antigen binding fragment thereof, and (iii) chemotherapy to the subject, wherein at least about 50% to about 90% of cells in a tumor sample obtained from the human subject have a staining intensity of 1, 2, or 3, as determined by IHC; and wherein the oleclumab or antigen binding fragment is administered to the subject at a dose of 1500 mg every 2 weeks for four doses and then every 4 weeks in a treatment cycle; the durvalumab or antigen binding fragment thereof is administered to the subject at a dose of 1500 mg every four weeks in the treatment cycle; and the chemotherapy comprises mFOLFOX and is administered on days 1 and 15 of the 28-day treatment cycle and then the cycle is repeated every 4 weeks.

In some aspects, the methods disclosed herein comprise administering (i) oleclumab or antigen binding fragment thereof, (ii) durvalumab or antigen binding fragment thereof, and (iii) chemotherapy to the subject, wherein at least about 50% to about 90% of cells in a tumor sample obtained from the human subject have a staining intensity of 1, 2, or 3, as determined by IHC; and wherein the oleclumab or antigen binding fragment is administered to the subject at a dose of 2250 mg every 2 weeks for four doses and then every 4 weeks in a treatment cycle; the durvalumab or antigen binding fragment thereof is administered to the subject at a dose of 1500 mg every four weeks in the treatment cycle; and the chemotherapy comprises mFOLFOX and is administered on days 1 and 15 of the 28-day treatment cycle and then the cycle is repeated every 4 weeks.

In some aspects, the methods disclosed herein comprise administering (i) oleclumab or antigen binding fragment thereof, (ii) durvalumab or antigen binding fragment thereof, and (iii) chemotherapy to the subject, wherein at least about 50% to about 90% of cells in a tumor sample obtained from the human subject have a staining intensity of 1, 2, or 3, as determined by IHC; and wherein the oleclumab or antigen binding fragment is administered to the subject at a dose of 3000 mg every 2 weeks for four doses and then every 4 weeks in a treatment cycle; the durvalumab or antigen binding fragment thereof is administered to the subject at a dose of 1500 mg every four weeks in the treatment cycle; and the chemotherapy comprises mFOLFOX and is administered on days 1 and 15 of the 28-day treatment cycle and then the cycle is repeated every 4 weeks.

In some aspects of the methods disclosed herein, at least about 30% to about 70% of cells in a tumor sample obtained from the human subject have a staining intensity of at least 2.

In some aspects, at least about 30%, about 35%, about 40%, about 45%, about 50%, about 55%, about 60%, about 65%, or about 70% of cells in a tumor sample obtained from the human subject have a staining intensity of at least 2.

In some aspects of the methods disclosed herein, the tumor is a 1st line metastatic pancreatic ductal adenocarcinoma. In some aspects, the tumor is a 2nd line metastatic pancreatic ductal adenocarcinoma.

### 6. BRIEF DESCRIPTION OF THE FIGURES

FIG. 1 shows the study design of the Phase 1b/2 study to evaluate the safety, pharmacokinetics, and clinical activity of MEDI9447 with or without durvalumab in combination with chemotherapy in subjects with metastatic pancreatic ductal adenocarcinoma.

FIG. 2A shows the treatment regimen for the dose escalation part (Part 1) of the Phase 1b/2 study.

FIG. 2B shows the treatment regimen for Cohort A (Arms A1, A2, and A3) in the dose expansion part (Part 2) of the Phase 1b/2 study.

FIG. 2C shows the treatment regimen for Cohort B (Arms B1, B2, and B3) in the dose expansion part (Part 2) of the Phase 1b/2 study.

FIG. 3 shows the progression free survival by CD73 levels in patients with low CD73 and high CD73 over time (in months) and the number of patients in each cohort and study arm. Arm A1 is gemcitabine/nab-paclitaxel; Arm A2 is oleclumab and gemcitabine/nab-paclitaxel; Arm A3 is oleclumab, durvalumab, and gemcitabine/nab-paclitaxel.

FIG. 4 shows the overall survival by study arms over time in months and the number of patients in each study arm. Arm A1 is gemcitabine/nab-paclitaxel; Arm A2 is oleclumab and gemcitabine/nab-paclitaxel; Arm A3 is oleclumab, durvalumab, and gemcitabine/nab-paclitaxel. Addition of oleclumab+durvalumab and chemotherapy leads to overall survival benefit in CD73 high population.

FIG. 5A shows the progression free survival by CD73 levels in patients with low CD73 and high CD73 over time (in months) and the number of patients in each cohort and study arm. Arm A1 is gemcitabine/nab-paclitaxel; Arm A2 is oleclumab and gemcitabine/nab-paclitaxel; Arm A3 is oleclumab, durvalumab, and gemcitabine/nab-paclitaxel.

FIG. 5B shows the overall survival by CD73 levels in patients with low CD73 and high CD73 over time (in months) and the number of patients in each cohort and study arm. Arm A1 is gemcitabine/nab-paclitaxel; Arm A2 is oleclumab and gemcitabine/nab-paclitaxel; Arm A3 is oleclumab, durvalumab, and gemcitabine/nab-paclitaxel.

FIG. 6A shows the overall survival by study arms over time in months and the number of patients in each study arm, irrespective of CD73 levels. Arm A1 is gemcitabine/nab-paclitaxel; Arm A2 is oleclumab and gemcitabine/nab-paclitaxel; Arm A3 is oleclumab, durvalumab, and gemcitabine/nab-paclitaxel.

FIG. 6B shows the overall survival by study arms over time in months and the number of patients in each study arm in patients that are in the CD73 high subgroup. Arm A1 is gemcitabine/nab-paclitaxel; Arm A2 is oleclumab and gemcitabine/nab-paclitaxel; Arm A3 is oleclumab, durvalumab, and gemcitabine/nab-paclitaxel. Addition of oleclumab+durvalumab and chemotherapy leads to overall survival benefit in CD73 high population.

FIG. 7A shows the progression free survival by study arms over time in months and the number of patients in each study arm, irrespective of CD73 levels. Arm A1 is gemcitabine/nab-paclitaxel; Arm A2 is oleclumab and gemcitabine/nab-paclitaxel; Arm A3 is oleclumab, durvalumab, and gemcitabine/nab-paclitaxel.

FIG. 7B shows the progression free survival by study arms over time in months and the number of patients in each study arm in patients that are in the CD73 high subgroup. Arm A1 is gemcitabine/nab-paclitaxel; Arm A2 is oleclumab and gemcitabine/nab-paclitaxel; Arm A3 is oleclumab, durvalumab, and gemcitabine/nab-paclitaxel. Addition of oleclumab+durvalumab and chemotherapy leads to progression free survival benefit in CD73 high population at 6 months.

### 7. DETAILED DESCRIPTION

In order that the present disclosure may be more readily understood, certain terms are first defined. As used in this application, except as otherwise expressly provided herein, each of the following terms shall have the meaning set forth below. Additional definitions are set forth throughout the application.

### 7.1 Definitions

An “antibody” (Ab) shall include, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region is comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. A heavy chain may have the C-terminal lysine or not. Unless specified otherwise herein, the amino acids in the variable regions are numbered using the Kabat numbering system and those in the constant regions are numbered using the EU system. In one embodiment, an antibody is a full-length antibody.

An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, monoclonal and polyclonal antibodies; chimeric and humanized antibodies; human or nonhuman antibodies; wholly synthetic antibodies; and single chain antibodies. A nonhuman antibody may be humanized by recombinant methods to reduce its immunogenicity in man.

As used herein, an “IgG antibody” has the structure of a naturally occurring IgG antibody, i.e., it has the same number of heavy and light chains and disulfide bonds as a naturally occurring IgG antibody of the same subclass. For example, an anti-CD73 IgG1, IgG2, IgG3 or IgG4 antibody consists of two heavy chains (HCs) and two light chains (LCs), wherein the two heavy chains and light chains are linked by the same number and location of disulfide bridges that occur in native IgG1, IgG2, IgG3 and IgG4 antibodies, respectively (unless the antibody has been mutated to modify the disulfide bonds)

An “isolated antibody” refers to an antibody that is substantially free of other antibodies having different antigenic specificities (e.g., an isolated antibody that binds specifically to CD73 is substantially free of antibodies that do not bind specifically to CD73). An isolated antibody that binds specifically to CD73 may, however, have cross-reactivity to other antigens, such as CD73 molecules from different species. Moreover, an isolated antibody may be substantially free of other cellular material and/or chemicals.

The antibody may be an antibody that has been altered (e.g., by mutation, deletion, substitution, conjugation to a non-antibody moiety). For example, an antibody may include one or more variant amino acids (compared to a naturally occurring antibody) which change a property (e.g., a functional property) of the antibody. For example, numerous such alterations are known in the art which affect, e.g., half-life, effector function, and/or immune responses to the antibody in a patient. The term antibody also includes artificial polypeptide constructs which comprise at least one antibody-derived antigen binding site.

The term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A mAb is an example of an isolated antibody. MAbs may be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region is also derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies are used synonymously.

A “humanized antibody” refers to an antibody in which some, most or all of the amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen” antibody refers to an antibody that binds specifically to the antigen. For example, an anti-CD73 antibody binds specifically to CD73.

An “antigen-binding portion” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments or portions of a full-length antibody. Examples of binding fragments encompassed within the term “antigen-binding portion” or “antigen-binding fragment” of an antibody, e.g., an anti-CD73 antibody described herein, include:
(1) a Fab fragment (fragment from papain cleavage) or a similar monovalent fragment consisting of the VL, VH, LC and CH1 domains;
(2) a F(ab′)2 fragment (fragment from pepsin cleavage) or a similar bivalent fragment comprising two Fab fragments linked by a disulfide bridge at the hinge region;
(3) a Fd fragment consisting of the VH and CH1 domains;
(4) a Fv fragment consisting of the VL and VH domains of a single arm of an antibody,
(5) a single domain antibody (dAb) fragment (Ward et al., (1989) Nature 341:544-46), which consists of a VH domain;
(6) a bi-single domain antibody which consists of two VH domains linked by a hinge (dual-affinity re-targeting antibodies (DARTs));
(7) a dual variable domain immunoglobulin;
(8) an isolated complementarity determining region (CDR); and
(9) a combination of two or more isolated CDRs, which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” or “antigen-binding fragment” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins. In some embodiments, an antibody is an antigen-binding fragment.

The terms “oleclumab” and “MEDI9447” as used herein refer to a human immunoglobulin G1 lambda (IgG1k) mAb that selectively binds to and inhibits the ectonucleotidase activity of CD73, as disclosed in U.S. Pat. No. 9,938,356, which is incorporated by reference herein in its entirety. The triple mutation, L234F/L235E/P331S (according to European Union numbering convention), is encoded in the heavy chain constant region to significantly reduce IgG effector function. Oleclumab inhibits the catalysis of AMP to adenosine and organic phosphate by CD73. Extracellular adenosine mediates the immunosuppressive effects of both MDSCs and Tregs, among others.

In particular embodiments, oleclumab or an antigen binding fragment thereof comprises a heavy chain variable domain and a light chain variable domain. In particular embodiments, oleclumab comprises a light chain variable domain comprising the amino acid sequence of SEQ ID NO: 1 and a heavy chain variable domain comprising the amino acid sequence of SEQ ID NO: 2. In other embodiments, oleclumab or an antigen-binding fragment thereof comprises a heavy chain variable domain and a light chain variable domain, wherein the heavy chain variable domain comprises CDR1, CDR2, and CDR3 sequences of SEQ ID NOs: 3-5, and wherein the light chain variable domain comprises CDR1, CDR2, and CDR3 sequences of SEQ ID NOs: 6-8.

The term “durvalumab” as used herein refers to an antibody that selectively binds PD-L1 and blocks the binding of PD-L1 to the PD-1 and CD80 receptors, as disclosed in U.S. Pat. No. 9,493,565, which is incorporated by reference herein in its entirety. The fragment crystallizable (Fc) domain of durvalumab contains a triple mutation in the constant domain of the IgG1 heavy chain that reduces binding to the complement component C1q and the Fcγ receptors responsible for mediating antibody-dependent cell-mediated cytotoxicity (ADCC).

In particular embodiments, durvalumab or an antigen-binding fragment thereof comprises a heavy chain variable domain and a light chain variable domain. In particular embodiments, durvalumab comprises a light chain variable domain comprising the amino acid sequence of SEQ ID NO: 9 and a heavy chain variable domain comprising the amino acid sequence of SEQ ID NO: 10. In other embodiments, durvalumab or an antigen-binding fragment thereof comprises a heavy chain variable domain and a light chain variable domain, wherein the heavy chain variable domain comprises CDR1, CDR2, and CDR3 sequences of SEQ ID NOs: 11-13, and wherein the light chain variable domain comprises CDR1, CDR2, and CDR3 sequences of SEQ ID NOs: 14-16.

A “patient” as used herein includes any patient who is afflicted with a cancer (e.g., hepatocellular carcinoma). The terms “subject” and “patient” are used interchangeably herein.

“Administering” refers to the physical introduction of a composition comprising a therapeutic agent to a subject, using any of the various methods and delivery systems known to those skilled in the art. Routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, in some embodiments, orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

“Treatment” or “therapy” of a subject refers to any type of intervention or process performed on, or the administration of an active agent to, the subject with the objective of reversing, alleviating, ameliorating, inhibiting, slowing down progression, development, severity or recurrence of a symptom, complication or condition, or biochemical indicia associated with a disease. Response Evaluation Criteria In Solid Tumors (RECIST) is a measure for treatment efficacy and are established rules that define when tumors respond, stabilize, or progress during treatment. RECIST 1.1 is the current guideline to solid tumor measurement and definitions for objective assessment of change in tumor size for use in adult and pediatric cancer clinical trials. Eastern Cooperative Oncology Group (ECOG) Performance Status is a numbering scale used to define the population of patients to be studied in a trial, so that it can be uniformly reproduced among physicians who enroll patients. In pediatric patients, the Lansky Performance Scale is a method for describing functional status in children. It was derived and internally validated in children with cancer to assess response to therapies and overall status.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. A beneficial effect can also take the form of arresting, slowing, retarding, or stabilizing of a deleterious progression of a marker of solid tumor. Effective treatment may refer to alleviation of at least one symptom of a solid tumor. Such effective treatment may, e.g., reduce patient pain, reduce the size and/or number of lesions, may reduce or prevent metastasis of a tumor, and/or may slow tumor growth.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to delay other unwanted cell proliferation. In some embodiments, an effective amount is an amount sufficient to prevent or delay tumor recurrence. An effective amount can be administered in one or more administrations. The effective amount of the drug or composition may: (i) reduce the number of cancer cells; (ii) reduce tumor size; (iii) inhibit, retard, slow to some extent and may stop cancer cell infiltration into peripheral organs; (iv) inhibit (i.e., slow to some extent and may stop tumor metastasis; (v) inhibit tumor growth; (vi) prevent or delay occurrence and/or recurrence of tumor; and/or (vii) relieve to some extent one or more of the symptoms associated with the cancer.

As used herein, the terms “combination” or “administered in combination” means that an antibody or antigen binding fragment thereof described herein can be administered with one or more additional therapeutic agents. In some aspects, an antibody or antigen binding fragment thereof can be administered with one or more additional therapeutic agents either simultaneously or sequentially. In some aspects, an antibody or antigen binding fragment thereof described herein can be administered with one or more additional therapeutic agent in the same or in different compositions.

The term “weight based dose” as referred to herein means that a dose that is administered to a patient is calculated based on the weight of the patient.

The term “progression-free survival,” which can be abbreviated as PFS, as used herein refers to the length of time during and after the treatment of a solid tumor (i.e., hepatocellular carcinoma) that a patient lives with the disease but it does not get worse.

“Dosing interval,” as used herein, means the amount of time that elapses between multiple doses of a formulation disclosed herein being administered to a subject. Dosing interval can thus be indicated as ranges.

The term “dosing frequency” as used herein refers to the frequency of administering doses of a formulation disclosed herein in a given time. Dosing frequency can be indicated as the number of doses per a given time, e.g., once a week or once in two weeks, etc.

The terms “about once a week,” “once about every week,” “once about every two weeks,” or any other similar dosing interval terms as used herein means approximate number, and “about once a week” or “once about every week” can include every seven days ±two days, i.e., every five days to every nine days. The dosing frequency of “once a week” thus can be every five days, every six days, every seven days, every eight days, or every nine days. “Once about every four weeks” can include every 28 days±3 days, i.e., every 25 days to every 31 days. Similar approximations apply, for example, to once about every three weeks, once about every four weeks, once about every five weeks, once about every six weeks and once about every twelve weeks. In some embodiments, a dosing interval of once about every four weeks means that the first dose can be administered any day in the first week, and then the next dose can be administered any day in fourth week. In other embodiments, a dosing interval of once about every four weeks means that the first dose is administered on a particular day of the first week (e.g., Monday) and then the next dose is administered on the same day of the fourth week (i.e., Monday), respectively.

A “cancer” refers a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor.

The term “tumor” as used herein refers to any mass of tissue that results from excessive cell growth or proliferation, either benign (non-cancerous) or malignant (cancerous), including pre-cancerous lesions.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including antibodies, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

A “tumor-infiltrating inflammatory cell” or “tumor-associated inflammatory cell” is any type of cell that typically participates in an inflammatory response in a subject and which infiltrates tumor tissue. Such cells include tumor-infiltrating lymphocytes (TILs), macrophages, monocytes, eosinophils, histiocytes and dendritic cells.

The use of the alternative (e.g., “or”) should be understood to mean either one, both, or any combination thereof of the alternatives. As used herein, the indefinite articles “a” or “an” should be understood to refer to “one or more” of any recited or enumerated component.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% or 20% (i.e., +10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one-tenth and one-hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary Of Biochemistry And Molecular Biology, 2006, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

Various aspects of the invention are described in further detail in the following subsections.

### 7.2 Methods of the Invention

In some aspects, the present disclosure is directed to a method for treating a metastatic pancreatic ductal adenocarcinoma (PDAC) in a subject in need thereof. A therapy comprising an anti-CD73 antibody or antigen binding fragment thereof results in better therapeutic outcomes (e.g., objective response rate and disease control rate) for afflicted subjects.

In one aspect, the disclosure includes a method of selecting a PDAC tumor in a human patient for immunotherapy, comprising determining the level of CD73 expression in a tumor sample. In some aspects, a tumor sample obtained from the subject expresses CD73.

In some aspects, the disclosure provides a method of treating a metastatic pancreatic ductal adenocarcinoma (PDAC) in a subject, the method comprising administering to the subject about 750 mg to about 3000 mg of an anti-CD73 antibody or antigen binding fragment thereof and chemotherapy. In one aspect, the invention includes a method of inhibiting the growth of a PDAC tumor in a subject, the method comprising administering to the subject about 750 mg to about 3000 mg of an anti-CD73 antibody or antigen binding fragment thereof and chemotherapy. In some aspects, the anti-CD73 antibody or antigen binding fragment thereof is oleclumab, i.e., MEDI9447. Oleclumab is a human immunoglobulin G1 lambda (IgG1k) mAb that selectively binds to and inhibits the ectonucleotidase activity of CD73, as disclosed in U.S. Pat. No. 9,938,356, which is incorporated by reference herein in its entirety. The triple mutation, L234F/L235E/P331S (according to European Union numbering convention), is encoded in the heavy chain constant region to significantly reduce IgG effector function.

In some aspects, the method of treating a metastatic pancreatic ductal adenocarcinoma (PDAC) in a subject comprises administering to the subject about 750 mg to about 3000 mg of an anti-CD73 antibody or antigen binding fragment thereof (for example, oleclumab). In some aspects, the method comprises administering about 700 mg, about 750 mg, about 800 mg, about 850 mg, about 900 mg, about 950 mg, about 1000 mg, about 1050 mg, about 1100 mg, about 1150 mg, about 1250 mg, about 1300 mg, about 1350 mg, about 1400 mg, about 1550 mg, about 1600 mg, about 1650 mg, about 1700 mg, about 1750 mg, about 1800 mg, about 1850 mg, about 1900 mg, about 1950 mg, about 2000 mg, about 2050 mg, about 2200 mg, about 2250 mg, about 2500 mg, about 2750 mg, or about 3500 mg.

In some aspects, the method of treating a metastatic pancreatic ductal adenocarcinoma (PDAC) in a subject comprises administering oleclumab or an antigen-binding fragment thereof at a dose of about 750 mg. In some aspects, the method of treating a metastatic pancreatic ductal adenocarcinoma (PDAC) in a subject comprises administering oleclumab or an antigen-binding fragment thereof at a dose of about 1500 mg. In some aspects, the method of treating a metastatic pancreatic ductal adenocarcinoma (PDAC) in a subject comprises administering oleclumab or an antigen-binding fragment thereof at a dose of about 2250 mg. In some aspects, the method of treating a metastatic pancreatic ductal adenocarcinoma (PDAC) in a subject comprises administering oleclumab or an antigen-binding fragment thereof at a dose of about 3000 mg.

In some aspects, the method of treating a metastatic pancreatic ductal adenocarcinoma (PDAC) in a subject comprises administering a dose of the oleclumab or antigen-binding fragment thereof to the subject once per treatment cycle. In some aspects, a treatment cycle is one, two, three, four, five, or six weeks. In some aspects, a treatment cycle is two weeks. In some aspects, a treatment cycle is four weeks. In some aspects, a treatment cycle is 28 days. In some aspects, a dose of antibody or antigen-binding fragment thereof described herein, e.g., oleclumab or an antigen-binding fragment thereof, is administered every two weeks for 4 doses, then every four weeks. In some aspects, the chemotherapy is administered on days 1, 8, and 15, of a 28-day cycle and then the cycle is repeated every 4 weeks.

In some aspects, the oleclumab or antigen-binding fragment thereof is administered to the subject every 14 to 28 days. In some aspects, the oleclumab or antigen-binding fragment thereof is administered to the subject every 14 days. In some aspects, the oleclumab or antigen-binding fragment thereof is administered to the subject every 21 days. In some aspects, the oleclumab or antigen-binding fragment thereof is administered to the subject every 28 days.

In some aspects, the method of treating a metastatic pancreatic ductal adenocarcinoma (PDAC) in a subject comprises administering a dose of oleclumab or an antigen-binding fragment thereof in combination with one or more chemotherapeutic agents.

In some aspects, the chemotherapeutic agent comprises gemcitabine. In some aspects, the chemotherapeutic agent comprises nab-paclitaxel. In some aspects, the chemotherapy comprises a combination of gemcitabine and nab-paclitaxel.

In some aspects, the gemcitabine is administered at a dose of about 1000 mg/m². In some aspects, the nab-paclitaxel is administered at a dose of about 125 mg/m. In some aspects, the gemcitabine is administered at a dose of 1000 mg/m². In some aspects, the nab-paclitaxel is administered at a dose of 125 mg/m².

In some aspects, the chemotherapeutic agent comprises a modified FOLFOX regimen (referred to herein as: mFOLFOX) comprising oxaliplatin, leucovorin, and 5-fluorouracil (5-FU). In some aspects, the mFOLFOX comprises oxaliplatin administered at a dose of about 85 mg/m², leucovorin administered at a dose of about 400 mg/m², and 5-FU administered at a dose of about 400 mg/m² followed by administration of a second dose of 5-FU at about 2400 mg/m². In some aspects, mFOLFOX is administered as follows: intravenous administration of oxaliplatin at a dose of about 85 mg/m², intravenous administration of leucovorin at a dose of about 400 mg/m², intravenous administration of a bolus of 5-FU at a dose of about 400 mg/m², followed by continuous intravenous infusion of 5-FU at a dose of about 2400 mg/m² administered over 46 to 48 hours. The mFOLFOX regimen is administered on days 1 and 15 of a 28-day treatment cycle and repeated every 4 weeks. In some aspects, the subject receiving the mFOLFOX is previously untreated or has progressed following gemcitabine-based chemotherapy. In some aspects the subject has not been exposed to 5-FU, capecitabine, or oxaliplatin.

In some aspects, the chemotherapeutic agent comprises FOLFOX-4. In some aspects, FOLFOX-4 comprises the following regimen: Day 1: Oxaliplatin 85 mg/m² and leucovorin 200 mg/m², followed by 5-FU 400 mg/m² IV bolus, followed by 5-FU 600 mg/m² IV infusion. Day 2: Leucovorin 200 mg/m², followed by 5-FU 400 mg/m² IV bolus, followed by 5-FU 600 mg/m² IV infusion.

In some aspects, the chemotherapeutic agent comprises FOLFOX-6. In some aspects, FOLFOX-6 comprises the following regimen: Day 1-2: Oxaliplatin 100 mg/m², concurrent with leucovorin 400 mg/m² (or levoleucovorin 200 mg/m²), followed by Fluorouracil 5-FU 400 mg/m² IV bolus, followed by Fluorouracil 5-FU infusion (2400 mg/m² for first two cycles, increased to 3000 mg/m² in case of no toxicity >grade 1 during the first two cycles). Days 3-14: Rest days. The regimen further comprises antiemetic prophylaxis with 5-HT3-receptor antagonist.

In some aspects, the chemotherapeutic agent comprises modified FOLFOX-6 (mFOLFOX-6). In some aspects, modified FOLFOX-6 comprises the following regimen: Day 1: 85 mg/m² oxaliplatin, 400 mg/m² 5-fluorouracil and 200 mg/m² leucovorin administered biweekly by intravenous infusion, followed by the administration of 2400 mg/m² 5-fluorouracil by a continuous infusion.

In some aspects, the chemotherapeutic agent comprises FOLFOX-7. In some aspects, FOLFOX-7 comprises the following regimen: 1-leucovorin 200 mg/m² or dl-leucovorin 400 mg/m₂ followed by fluorouracil infusion of 2,400 mg/m² every 2 weeks, with oxaliplatin 130 mg/m² as a 2-hour infusion on day 1.

In some aspects, the chemotherapeutic agent comprises a FOLFIRINOX regimen. FOLFIRINOX comprises oxaliplatin, leucovorin, irinotecan, and 5-FU. FOLFIRINOX comprises oxaliplatin (85 mg/m², administered over 2 hours), followed by leucovorin (400 mg/m², administered over 2 hours), with the addition after 30 minutes of irinotecan (180 mg/m², administered over 90 minutes), followed by 5-FU (400 mg/m²) by intravenous bolus, on Day 1. Then, a continuous intravenous infusion of 5-FU (2400 mg/m²) is administered over 46 hours starting on Day 1. In some aspects, the mFOLFIRINOX regimen is administered on day 1 of a 2-week treatment cycle and repeated every 2 weeks. In some aspects, the subject receiving FOLFIRINOX is previously untreated or has progressed following gemcitabine-based chemotherapy. In some aspects the subject has not been exposed to 5-FU, capecitabine, or oxaliplatin.

In some aspects, the oleclumab or an antigen-binding fragment thereof and chemotherapy are administered concurrently. In some aspects, the oleclumab or an antigen-binding fragment thereof and chemotherapy are administered sequentially.

In one aspect, the method of treating a metastatic pancreatic ductal adenocarcinoma (PDAC) in a subject comprises administering oleclumab or an antigen-binding fragment thereof and chemotherapy, wherein oleclumab or antigen binding fragment thereof is administered at a dose of 750 to 3000 mg every 2 weeks for four doses and then every 4 weeks, and the chemotherapy comprises 1000 mg/m² gemcitabine and 125 mg/m² nab-paclitaxel, wherein the chemotherapy is administered on days 1, 8, and 15 of a 28-day cycles and then every 4 weeks. In some aspects, the oleclumab or antigen binding fragment and the chemotherapy can be administered simultaneously or sequentially.

In some aspects, the disclosure provides a method of treating a metastatic pancreatic ductal adenocarcinoma (PDAC) in a subject, the method comprising administering to the subject about 750 mg to about 3000 mg of an anti-CD73 antibody or antigen binding fragment thereof, e.g., oleclumab, and chemotherapy, further comprising administering to the subject about 1500 mg of an anti-PD-L1 antibody or antigen binding fragment thereof. In some aspects, the anti-PD-L1 antibody or antigen binding fragment thereof comprises durvalumab or antigen binding fragment thereof.

In some aspects, the durvalumab or antigen binding fragment thereof is administered at a dose of about 1500 mg. In some aspects, the durvalumab or antigen binding fragment thereof is administered at a dose of 1500 mg. In some aspects, the dose of durvalumab or antigen binding fragment thereof is administered every four weeks.

In some aspects, the method of treating a metastatic pancreatic ductal adenocarcinoma (PDAC) in a subject disclosed herein comprises administering to the subject about 750 mg to about 3000 mg of oleclumab or antigen binding fragment thereof and chemotherapy, wherein the oleclumab or antigen binding fragment thereof is administered at a dose of 1500 mg every 2 weeks for four doses and then every 4 weeks, and wherein the durvalumab or antigen binding fragment thereof is administered at a dose of 1500 mg every four weeks, and wherein the chemotherapy comprises 1000 mg/m² gemcitabine and 125 mg/m² nab-paclitaxel, wherein the chemotherapy is administered on days 1, 8, and 15 of a 28-day cycle and then every 4 weeks. In some aspects, the oleclumab or antigen binding fragment thereof, the durvalumab or antigen binding fragment thereof, and the chemotherapy can be administered simultaneously or sequentially.

In some aspects, the method of treating a metastatic pancreatic ductal adenocarcinoma (PDAC) in a subject disclosed herein comprises administering to the subject about 750 mg to about 3000 mg of oleclumab or antigen binding fragment thereof and chemotherapy, wherein the oleclumab or antigen binding fragment thereof is administered at a dose of 1500 mg every 2 weeks for four doses and then every 4 weeks, and wherein the durvalumab or antigen binding fragment thereof is administered at a dose of 1500 mg every four weeks, and wherein the chemotherapy comprises mFOLFOX, wherein the chemotherapy is administered on Days 1 and 15 of a 28-day cycle and then every 4 weeks. In some aspects, the oleclumab or antigen binding fragment thereof, the durvalumab or antigen binding fragment thereof, and the chemotherapy can be administered simultaneously or sequentially. In some aspects, the subject receiving the mFOLFOX is previously untreated or has progressed following gemcitabine-based chemotherapy. In some aspects the subject has not been exposed to 5-FU, capecitabine, or oxaliplatin.

In some aspects, the method of treating a metastatic pancreatic ductal adenocarcinoma (PDAC) in a subject disclosed herein comprises administering to the subject about 750 mg to about 3000 mg of oleclumab or antigen binding fragment thereof and chemotherapy, wherein the oleclumab or antigen binding fragment thereof is administered at a dose of 1500 mg every 2 weeks for four doses and then every 4 weeks, and wherein the durvalumab or antigen binding fragment thereof is administered at a dose of 1500 mg every four weeks, and wherein the chemotherapy comprises FOLFIRINOX, wherein the chemotherapy is administered on Day 1 of a 14-day cycle and then repeated every 2 weeks. In some aspects, the oleclumab or antigen binding fragment thereof, the durvalumab or antigen binding fragment thereof, and the chemotherapy can be administered simultaneously or sequentially. In some aspects, the subject receiving the FOLFIRINOX is previously untreated or has progressed following gemcitabine-based chemotherapy. In some aspects the subject has not been exposed to 5-FU, capecitabine, or oxaliplatin.

In some aspects, the method of treating a metastatic pancreatic ductal adenocarcinoma (PDAC) disclosed herein involves parental administration of an antibody or antigen-fragment thereof described herein. In some aspects, the administration is intravenous. In some aspects, the administration is via intravenous infusion.

### 7.3 Immunohistochemistry (IHC) Detection Method

In some aspects, the method of treating a metastatic pancreatic ductal adenocarcinoma (PDAC) in a subject disclosed herein comprises detecting the expression of CD73 using an immunohistochemistry (IHC) detection method. In some aspects, the IHC detection method is an automated IHC method. In some aspects of the IHC detection method described herein, the staining intensities are defined as 0, 1+, 2+, and 3+ intensities, with 0 indicating the lack of staining and 3 indicating strong staining. The IHC method is further described elsewhere herein.

In some aspects of the method of treating a metastatic pancreatic ductal adenocarcinoma (PDAC) in a subject disclosed herein, CD73 has been detected in at least about 70% of cells in a tumor sample from the subject prior to the administration.

In some aspects of the method of treating a metastatic pancreatic ductal adenocarcinoma (PDAC) in a subject disclosed herein, the method further comprises detecting CD73 in at least about 70% of cells in a tumor sample from the subject prior to the administration of an anti-CD73 antibody or antigen binding fragment thereof, e.g., oleclumab or antigen binding fragment thereof.

In some aspects, CD73 expression in tumor samples can be evaluated by immunohistochemistry.

In one aspect, cell surface CD73 staining intensity is evaluated and scored as 0, 1, 2, or 3 based on the intensity of staining.

In another aspect, cell surface CD73 staining is evaluated by calculating the percentage of the cells scored as 1, 2, or 3 cells within the tumor sample relative to cells in the tumor sample. In some aspects, the percentage is reported as a P-score. The P-score is the sum of the percentage of tumor cells demonstrating staining at 1, 2, or 3+ intensities. The P-score=(% at 1)+(% at 2)+(% at 3).

In another aspect, cell surface CD73 staining is evaluated by assessing the percentage of the cells scored as 2 or 3 cells within the tumor sample. The 2+3+P-score is the sum of the percentage of tumor cells staining at 2, or 3+ intensities. The 2+3+P-score=(% at 2)+(% at 3).

In some aspects, a tumor sample obtained from a subject for use in any of the methods disclosed herein has a P-score in a range of from at least about 50% to about 90%. In some aspects, the tumor sample has a P-score of at least about 50%, about 55%, about 60%, about 65%, about 70%, about 75%, about 80%, about 85%, or about 90%. In some aspects, the tumor sample has a P-score of at least about 50%, about 55%, about 60%, about 65%, about 70%, about 75%, about 80%, about 85%, or about 90%.

In some aspects, a tumor sample obtained from a human patient for use in any method disclosed herein has a 2+3+P-score in a range of from at least about 30% to about 70%. In some aspects, the tumor sample has a 2+3+P-score of at least about 30%, about 35%, about 40%, about 45%, about 50%, about 55%, about 60%, about 65%, or about 70%. In some aspects, the tumor sample has a 2+3+P-score of at least about 30%, about 35%, about 40%, about 45%, about 50%, about 55%, about 60%, about 65%, or about 70%.

Tumor samples from patients considered to have high CD73 expression have a P-score ranging from at least about 50% to about 90%. Tumor samples from patients considered to have high CD73 expression have a 2+3+P-score ranging from at least about 30% to about 70%.

### 7.4 Anti-CD73 Antibody and Antigen-Binding Fragments Thereof

Provided herein are methods of treating cancers in a subject (e.g., a human subject) comprising administering to the subject antibodies (e.g., monoclonal antibodies, such as chimeric, humanized, or human antibodies) and antigen-binding fragments thereof which specifically bind to CD73 (e.g., human CD73). In some aspects, CD73 (e.g., human CD73) antibodies and antigen-binding fragments thereof that can be used in the methods provided herein include MEDI9447, referred to herein as oleclumab,” which is a human immunoglobulin G1 lambda (IgG1k) mAb that selectively binds to and inhibits the ectonucleotidase activity of CD73. The Fc domain of MEDI9447 carries the triple mutation, L234F/L235E/P331S (according to European Union numbering convention), designed to reduce Fc-mediated immune effector functions. Oleclumab inhibits the catalysis of AP to adenosine and organic phosphate by CD73. Extracellular adenosine mediates the immunosuppressive effects of both MDSCs and Tregs, among others.

MEDI9447, i.e., oleclumab, is disclosed in U.S. Pat. No. 9,938,356, which is incorporated by reference herein in its entirety.

In some aspects of the methods disclosed herein, the method further comprises administering an anti-PD-L1 antibody or antigen binding fragment thereof to the subject (i.e., a human subject). In some aspects, PD-L1 (e.g., human PD-L1) antibodies and antigen-binding fragments thereof that can be used in the methods provided herein include durvalumab or an antigen binding fragment thereof. Durvalumab is an antibody that selectively binds PD-L1 and blocks the binding of PD-L1 to the PD-1 and CD80 receptors. The fragment crystallizable (Fc) domain of durvalumab contains a triple mutation in the constant domain of the IgG1 heavy chain that reduces binding to the complement component C1q and the Fcγ receptors responsible for mediating antibody-dependent cell-mediated cytotoxicity (ADCC).

Durvalumab is disclosed in U.S. Pat. No. 9,493,565, which is incorporated by reference herein in its entirety.

In some aspects of the present disclosure, an antibody or antigen-binding fragment thereof for use in the methods described herein specifically binds to human CD73 (e.g., oleclumab) or PD-L1 (e.g., durvalumab) and comprises the six CDRs of the oleclumab and durvalumab antibodies provided in Table 1.

TABLE 1
CDR Amino Acid Sequences of Oleclumab and Durvalumab

| Antibody | (SEQ ID NO:) | (SEQ ID NO:) | (SEQ ID NO:) |
|---|---|---|---|
| Oleclumab | SYAYS | AISGSGGRTYYADSVKG | LGYGRVDE |
| VH CDR1-3 | (SEQ ID NO: 3) | (SEQ ID NO: 4) | (SEQ ID NO: 5) |
| Oleclumab | SGSLSNIGRNPV | LDNLRLS | ATWDDSHPGWT |
| VL CDR1-3 | N | (SEQ ID NO: 7) | (SEQ ID NO: 8) |
| | (SEQ ID NO: 6) | | |
| Durvalumab | GFTFSRYWMS | NIKQDGSEKYYVDSVKG | EGGWFGELAFDY |
| VH CDR1-3 | (SEQ ID NO: 11) | (SEQ ID NO: 12) | (SEQ ID NO: 13) |
| Durvalumab | RASQRVSSSYLA | DASSRAT | QQYGSLPWT |
| VL CDR1-3 | (SEQ ID NO: 14) | (SEQ ID NO: 15) | (SEQ ID NO: 16) |

In some aspects of the present disclosure, an antibody or antigen-binding fragment thereof for use in the methods described herein specifically binds to human CD73 (e.g., oleclumab) or PD-L1 (e.g., durvalumab) and comprise the variable heavy chain (VH) and variable light chain (VL) sequences of the oleclumab and durvalumab antibodies, as set forth in Table 2.

TABLE 2
Variable light chain (VL) and variable heavy chain (VH) amino acid
sequences of oleclumab and durvalumab

| Antibody | Sequence | Description |
|---|---|---|
| Oleclumab | QSVLTQPPSASGTPGQRVTISCSGSLSNIGRNPVN | VL |
| | WYQQLPGTAPKLLIYLDNLRLSGVPDRFSGSKS | |
| | GTSASLAISGLQSEDEADYYCATWDDSHPGWTF | |
| | GGGTKLTVL (SEQ ID NO: 1) | |
| Oleclumab | EVQLLESGGGLVQPGGSLRLSCAASGFTFSSYA | VH |
| | YSWVRQAPGKGLEWVSAISGSGGRTYYADSVK | |
| | GRFTISRDNSKNTLYLQMNSLRAEDTAVYYCAR | |
| | LGYGRVDEWGRGTLVTVSS (SEQ ID NO: 2) | |
| Durvalumab | EIVLTQSPGTLSLSPGERATLSCRASQRVSSSYLA | VL |
| | WYQQKPGQAPRLLIYDASSRATGIPDRFSGSGS | |
| | GTDFTLTISRLEPEDFAVYYCQQYGSLPWTFGQ | |
| | GTKVEIK (SEQ ID NO: 9) | |
| Durvalumab | EVQLVESGGGLVQPGGSLRLSCAASGFTFSRYW | VH |
| | MSWVRQAPGKGLEWVANIKQDGSEKYYVDSV | |
| | KGRFTISRDNAKNSLYLQMNSLRAEDTAVYYC | |
| | AREGGWFGELAFDYWGQGTLVTVSS (SEQ ID | |
| | NO: 10) | |

In some aspects of the present disclosure, an anti-CD73 antibody or antigen binding fragment thereof comprises oleclumab, wherein the oleclumab comprises a heavy chain variable domain and a light chain variable domain, wherein the heavy chain variable domain comprises CDR1, CDR2, and CDR3 sequences of SEQ ID NOs: 3-5, and wherein the light chain variable domain comprises CDR1, CDR2, and CDR3 sequences of SEQ ID NOs: 6-8.

In some aspects of the present disclosure, an anti-CD73 antibody or antigen binding fragment thereof comprises oleclumab, wherein the oleclumab comprises a light chain variable domain comprising the amino acid sequence of SEQ ID NO: 1 and a heavy chain variable domain comprising the amino acid sequence of SEQ ID NO: 2.

In some aspects of the present disclosure, an anti-PD-L1 antibody or antigen binding fragment thereof comprises durvalumab, wherein the durvalumab comprises a heavy chain variable domain and a light chain variable domain, wherein the heavy chain variable domain comprises CDR1, CDR2, and CDR3 sequences of SEQ ID NOs: 11-13, and wherein the light chain variable domain comprises CDR1, CDR2, and CDR3 sequences of SEQ ID NOs: 14-16.

In some aspects of the present disclosure, an anti-PD-L1 antibody or antigen binding fragment thereof comprises durvalumab, wherein the durvalumab comprises a light chain variable domain comprising the amino acid sequence of SEQ ID NO: 9 and a heavy chain variable domain comprising the amino acid sequence of SEQ ID NO: 10.

In some aspects, provided herein are methods of treating a metastatic PDAC in a subject, the method comprising administering to the subject: (1) about 750 mg to about 3000 mg of an anti-CD73 antibody or antigen binding fragment thereof, wherein the anti-CD73 antibody or antigen binding fragment thereof comprises a light chain variable domain comprising the amino acid sequence of SEQ ID NO: 1 and a heavy chain variable domain comprising the amino acid sequence of SEQ ID NO: 2; and (2) chemotherapy, wherein the chemotherapy is gemcitabine administered at a dose of 1000 mg/m2 and nab-paclitaxel administered at a dose of 125 mg/m, wherein a tumor sample obtained from the subject comprises about 50% to about 90% of cells having an IHC staining intensity score of 1, 2, or 3.

In some aspects, provided herein are methods of treating a metastatic PDAC in a subject, the method comprising administering to the subject: (1) about 750 mg to about 3000 mg of an anti-CD73 antibody or antigen binding fragment thereof, wherein the anti-CD73 antibody or antigen binding fragment thereof comprises a light chain variable domain comprising the amino acid sequence of SEQ ID NO: 1 and a heavy chain variable domain comprising the amino acid sequence of SEQ ID NO: 2; and (2) chemotherapy, wherein the chemotherapy is gemcitabine administered at a dose of 1000 mg/m² and nab-paclitaxel administered at a dose of 125 mg/m², wherein a tumor sample obtained from the subject comprises at least about 30% to about 70% of cells having an IHC staining intensity score of at least 2.

In some aspects of the methods provided herein, an anti-CD73 antibody or antigen binding fragment thereof is administered every 2 weeks for four doses and then every 4 weeks, and wherein the chemotherapy is administered on days 1, 8, and 15 of a 28-day cycle and then repeated every 4 weeks. In some aspects, the anti-CD73 antibody or antigen binding fragment thereof, and the chemotherapy are administered simultaneously or sequentially.

In some aspects, provided herein are methods of treating a metastatic PDAC in a subject, the method comprising administering to the subject: (1) about 750 mg to about 3000 mg of an anti-CD73 antibody or antigen binding fragment thereof, wherein the anti-CD73 antibody or antigen binding fragment thereof comprises a light chain variable domain comprising the amino acid sequence of SEQ ID NO: 1 and a heavy chain variable domain comprising the amino acid sequence of SEQ ID NO: 2; (2) about 1500 mg of anti-PD-L1 antibody or antigen binding fragment thereof, wherein the anti-PD-L1 antibody or antigen binding fragment thereof comprises a light chain variable domain comprising the amino acid sequence of SEQ ID NO: 9 and a heavy chain variable domain comprising the amino acid sequence of SEQ ID NO: 10; and (3) chemotherapy, wherein the chemotherapy is gemcitabine administered at a dose of 1000 mg/m² and nab-paclitaxel administered at a dose of 125 mg/m², wherein a tumor sample obtained from the subject comprises at least about 50% to about 90% of cells having an IHC staining intensity score of 1, 2, or 3.

In some aspects, provided herein are methods of treating a metastatic PDAC in a subject, the method comprising administering to the subject: (1) about 750 mg to about 3000 mg of an anti-CD73 antibody or antigen binding fragment thereof, wherein the anti-CD73 antibody or antigen binding fragment thereof comprises a light chain variable domain comprising the amino acid sequence of SEQ ID NO: 1 and a heavy chain variable domain comprising the amino acid sequence of SEQ ID NO: 2; (2) about 1500 mg of anti-PD-L1 antibody or antigen binding fragment thereof, wherein the anti-PD-L1 antibody or antigen binding fragment thereof comprises a light chain variable domain comprising the amino acid sequence of SEQ ID NO: 9 and a heavy chain variable domain comprising the amino acid sequence of SEQ ID NO: 10; and (3) chemotherapy, wherein the chemotherapy is gemcitabine administered at a dose of 1000 mg/m² and nab-paclitaxel administered at a dose of 125 mg/m², wherein a tumor sample obtained from the subject comprises at least about 30% to about 70% of cells having an IHC staining intensity score of at least 2.

In some aspects, the anti-CD73 antibody or antigen binding fragment thereof is administered every 2 weeks for four doses and then every 4 weeks, the anti-PD-L1 antibody or antigen binding fragment thereof is administered every four weeks, and the chemotherapy is administered on days 1, 8, and 15 of a 28-day cycle and then repeated every 4 weeks. In some aspects, the anti-CD73 antibody or antigen binding fragment thereof, the anti-PD-L1 antibody or antigen binding fragment thereof, and the chemotherapy are administered simultaneously or sequentially.

In some aspects, provided herein are methods of treating a metastatic PDAC in a subject, the method comprising administering to the subject: (1) about 750 mg to about 3000 mg of an anti-CD73 antibody or antigen binding fragment thereof, wherein the anti-CD73 antibody or antigen binding fragment thereof comprises a light chain variable domain comprising the amino acid sequence of SEQ ID NO: 1 and a heavy chain variable domain comprising the amino acid sequence of SEQ ID NO: 2; and (2) chemotherapy, wherein the chemotherapy is mFOLFOX, wherein a tumor sample obtained from the subject comprises at least about 50% to about 90% of cells having an IHC staining intensity score of 1, 2, or 3. In some aspects, the subject receiving the mFOLFOX is previously untreated or has progressed following gemcitabine-based chemotherapy. In some aspects the subject has not been exposed to 5-FU, capecitabine, or oxaliplatin.

In some aspects, provided herein are methods of treating a metastatic PDAC in a subject, the method comprising administering to the subject: (1) about 750 mg to about 3000 mg of an anti-CD73 antibody or antigen binding fragment thereof, wherein the anti-CD73 antibody or antigen binding fragment thereof comprises a light chain variable domain comprising the amino acid sequence of SEQ ID NO: 1 and a heavy chain variable domain comprising the amino acid sequence of SEQ ID NO: 2; and (2) chemotherapy, wherein the chemotherapy mFOLFOX, wherein a tumor sample obtained from the subject comprises at least about 30% to about 70% of cells having an IHC staining intensity score of at least 2. In some aspects, the subject receiving the mFOLFOX is previously untreated or has progressed following gemcitabine-based chemotherapy. In some aspects the subject has not been exposed to 5-FU, capecitabine, or oxaliplatin.

In some aspects of the methods provided herein, an anti-CD73 antibody or antigen binding fragment thereof is administered every 2 weeks for four doses and then every 4 weeks, and wherein the chemotherapy is administered on days 1 and 15 of a 28-day cycle and then repeated every 4 weeks. In some aspects, the anti-CD73 antibody or antigen binding fragment thereof, and the chemotherapy are administered simultaneously or sequentially.

In some aspects, provided herein are methods of treating a metastatic PDAC in a subject, the method comprising administering to the subject: (1) about 750 mg to about 3000 mg of an anti-CD73 antibody or antigen binding fragment thereof, wherein the anti-CD73 antibody or antigen binding fragment thereof comprises a light chain variable domain comprising the amino acid sequence of SEQ ID NO: 1 and a heavy chain variable domain comprising the amino acid sequence of SEQ ID NO: 2; (2) about 1500 mg of anti-PD-L1 antibody or antigen binding fragment thereof, wherein the anti-PD-L1 antibody or antigen binding fragment thereof comprises a light chain variable domain comprising the amino acid sequence of SEQ ID NO: 9 and a heavy chain variable domain comprising the amino acid sequence of SEQ ID NO: 10; and (3) chemotherapy, wherein the chemotherapy is mFOLFOX, wherein a tumor sample obtained from the subject comprises at least about 50% to about 90% of cells having an IHC staining intensity score of 1, 2, or 3. In some aspects, the subject receiving the mFOLFOX is previously untreated or has progressed following gemcitabine-based chemotherapy. In some aspects the subject has not been exposed to 5-FU, capecitabine, or oxaliplatin.

In some aspects, provided herein are methods of treating a metastatic PDAC in a subject, the method comprising administering to the subject: (1) about 750 mg to about 3000 mg of an anti-CD73 antibody or antigen binding fragment thereof, wherein the anti-CD73 antibody or antigen binding fragment thereof comprises a light chain variable domain comprising the amino acid sequence of SEQ ID NO: 1 and a heavy chain variable domain comprising the amino acid sequence of SEQ ID NO: 2; (2) about 1500 mg of anti-PD-L1 antibody or antigen binding fragment thereof, wherein the anti-PD-L1 antibody or antigen binding fragment thereof comprises a light chain variable domain comprising the amino acid sequence of SEQ ID NO: 9 and a heavy chain variable domain comprising the amino acid sequence of SEQ ID NO: 10; and (3) chemotherapy, wherein the chemotherapy is mFOLFOX, wherein a tumor sample obtained from the subject comprises at least about 30% to about 70% of cells having an IHC staining intensity score of at least 2. In some aspects, the subject receiving the mFOLFOX is previously untreated or has progressed following gemcitabine-based chemotherapy. In some aspects the subject has not been exposed to 5-FU, capecitabine, or oxaliplatin.

In some aspects, the anti-CD73 antibody or antigen binding fragment thereof is administered every 2 weeks for four doses and then every 4 weeks, the anti-PD-L1 antibody or antigen binding fragment thereof is administered every four weeks, and the chemotherapy is administered on days 1 and 15 of a 28-day cycle and then repeated every 4 weeks. In some aspects, the anti-CD73 antibody or antigen binding fragment thereof, the anti-PD-L1 antibody or antigen binding fragment thereof, and the chemotherapy are administered simultaneously or sequentially.

In some aspects, provided herein are methods of treating a metastatic PDAC in a subject, the method comprising administering to the subject: (1) about 750 mg to about 3000 mg of an anti-CD73 antibody or antigen binding fragment thereof, wherein the anti-CD73 antibody or antigen binding fragment thereof comprises a light chain variable domain comprising the amino acid sequence of SEQ ID NO: 1 and a heavy chain variable domain comprising the amino acid sequence of SEQ ID NO: 2; and (2) chemotherapy, wherein the chemotherapy is FOLFIRINOX, wherein a tumor sample obtained from the subject comprises at least about 50% to about 90% of cells having an IHC staining intensity score of 1, 2, or 3. In some aspects, the subject receiving the FOLFIRINOX is previously untreated or has progressed following gemcitabine-based chemotherapy. In some aspects the subject has not been exposed to 5-FU, capecitabine, or oxaliplatin.

In some aspects, provided herein are methods of treating a metastatic PDAC in a subject, the method comprising administering to the subject: (1) about 750 mg to about 3000 mg of an anti-CD73 antibody or antigen binding fragment thereof, wherein the anti-CD73 antibody or antigen binding fragment thereof comprises a light chain variable domain comprising the amino acid sequence of SEQ ID NO: 1 and a heavy chain variable domain comprising the amino acid sequence of SEQ ID NO: 2; and (2) chemotherapy, wherein the chemotherapy is FOLFIRINOX, wherein a tumor sample obtained from the subject comprises at least about 30% to about 70% of cells having an IHC staining intensity score of at least 2. In some aspects, the subject receiving the FOLFIRINOX is previously untreated or has progressed following gemcitabine-based chemotherapy. In some aspects the subject has not been exposed to 5-FU, capecitabine, or oxaliplatin.

In some aspects of the methods provided herein, an anti-CD73 antibody or antigen binding fragment thereof is administered every 2 weeks for four doses and then every 4 weeks, and wherein the chemotherapy is administered on day 1 of a 14-day cycle and then repeated every 2 weeks. In some aspects, the anti-CD73 antibody or antigen binding fragment thereof, and the chemotherapy are administered simultaneously or sequentially.

In some aspects, provided herein are methods of treating a metastatic PDAC in a subject, the method comprising administering to the subject: (1) about 750 mg to about 3000 mg of an anti-CD73 antibody or antigen binding fragment thereof, wherein the anti-CD73 antibody or antigen binding fragment thereof comprises a light chain variable domain comprising the amino acid sequence of SEQ ID NO: 1 and a heavy chain variable domain comprising the amino acid sequence of SEQ ID NO: 2; (2) about 1500 mg of anti-PD-L1 antibody or antigen binding fragment thereof, wherein the anti-PD-L1 antibody or antigen binding fragment thereof comprises a light chain variable domain comprising the amino acid sequence of SEQ ID NO: 9 and a heavy chain variable domain comprising the amino acid sequence of SEQ ID NO: 10; and (3) chemotherapy, wherein the chemotherapy is FOLFIRINOX, wherein a tumor sample obtained from the subject comprises at least about 50% to about 90% of cells having an IHC staining intensity score of 1, 2, or 3. In some aspects, the subject receiving the FOLFIRINOX is previously untreated or has progressed following gemcitabine-based chemotherapy. In some aspects the subject has not been exposed to 5-FU, capecitabine, or oxaliplatin.

In some aspects, provided herein are methods of treating a metastatic PDAC in a subject, the method comprising administering to the subject: (1) about 750 mg to about 3000 mg of an anti-CD73 antibody or antigen binding fragment thereof, wherein the anti-CD73 antibody or antigen binding fragment thereof comprises a light chain variable domain comprising the amino acid sequence of SEQ ID NO: 1 and a heavy chain variable domain comprising the amino acid sequence of SEQ ID NO: 2; (2) about 1500 mg of anti-PD-L1 antibody or antigen binding fragment thereof, wherein the anti-PD-L1 antibody or antigen binding fragment thereof comprises a light chain variable domain comprising the amino acid sequence of SEQ ID NO: 9 and a heavy chain variable domain comprising the amino acid sequence of SEQ ID NO: 10; and (3) chemotherapy, wherein the chemotherapy is FOLFIRINOX, wherein a tumor sample obtained from the subject comprises at least about 30% to about 70% of cells having an IHC staining intensity score of at least 2. In some aspects, the subject receiving the FOLFIRINOX is previously untreated or has progressed following gemcitabine-based chemotherapy. In some aspects the subject has not been exposed to 5-FU, capecitabine, or oxaliplatin.

In some aspects, the anti-CD73 antibody or antigen binding fragment thereof is administered every 2 weeks for four doses and then every 4 weeks, the anti-PD-L1 antibody or antigen binding fragment thereof is administered every four weeks, and the chemotherapy is administered on day 1 of a 14-day cycle and then repeated every 2 weeks. In some aspects, the anti-CD73 antibody or antigen binding fragment thereof, the anti-PD-L1 antibody or antigen binding fragment thereof, and the chemotherapy are administered simultaneously or sequentially.

In some aspects, provided herein are methods of inhibiting the growth of a tumor in a human subject comprising administering (i) oleclumab or antigen binding fragment thereof and (ii) chemotherapy to the subject, wherein at least about 50% to about 90% of cells in a tumor sample obtained from the human subject have a staining intensity of 1, 2, or 3, as determined by IHC; and wherein the oleclumab or antigen binding fragment is administered to the subject at a dose of 750 mg to 3000 mg every 2 weeks for four doses and then every 4 weeks; and the chemotherapy comprises 1000 mg/m² gemcitabine and 125 mg/m² nab-paclitaxel and is administered on days 1, 8, and 15 of a 28-day cycle and then the cycle is repeated every 4 weeks. In some aspects, the oleclumab or antigen binding fragment is administered to the subject at a dose of 750 mg. In some aspects, the oleclumab or antigen binding fragment is administered to the subject at a dose of 1500 mg. In some aspects, the oleclumab or antigen binding fragment is administered to the subject at a dose of 2250 mg. In some aspects, the oleclumab or antigen binding fragment is administered to the subject at a dose of 3000 mg.

In some aspects of the methods of inhibiting the growth of a tumor in a human subject comprising administering (i) oleclumab or antigen binding fragment thereof described herein, at least about 30% to about 70% of cells in a tumor sample obtained from the human subject have a staining intensity of at least 2.

In some aspects, provided herein are methods of inhibiting the growth of a tumor in a human subject comprising administering (i) oleclumab or antigen binding fragment thereof, (ii) durvalumab or antigen binding fragment thereof, and (iii) chemotherapy to the subject, wherein at least about 50% to about 90% of cells in a tumor sample obtained from the human subject have a staining intensity of 1, 2, or 3, as determined by IHC, and the oleclumab or antigen binding fragment is administered to the subject at a dose of 750 mg to 3000 mg every 2 weeks for four doses and then every 4 weeks; the durvalumab or antigen binding fragment thereof is administered to the subject at a dose of 1500 mg every four weeks; and the chemotherapy comprises 1000 mg/m² gemcitabine and 125 mg/m² nab-paclitaxel administered on days 1, 8, and 15 of a 28-day cycle and then the cycle is repeated every 4 weeks. In some aspects, the oleclumab or antigen binding fragment is administered to the subject at a dose of 750 mg. In some aspects, the oleclumab or antigen binding fragment is administered to the subject at a dose of 1500 mg. In some aspects, the oleclumab or antigen binding fragment is administered to the subject at a dose of 2250 mg. In some aspects, the oleclumab or antigen binding fragment is administered to the subject at a dose of 3000 mg.

In some aspects of the methods of inhibiting the growth of a tumor in a human subject comprising administering (i) oleclumab or antigen binding fragment thereof described herein, at least about 30% to about 70% of cells in a tumor sample obtained from the human subject have a staining intensity of at least 2.

In some aspects, provided herein are methods of inhibiting the growth of a tumor in a human subject comprising administering (i) oleclumab or antigen binding fragment thereof and (ii) chemotherapy to the subject, wherein at least about 50% to about 90% of cells in a tumor sample obtained from the human subject have a staining intensity of 1, 2, or 3, as determined by IHC; and wherein the oleclumab or antigen binding fragment is administered to the subject at a dose of 750 mg to 3000 mg every 2 weeks for four doses and then every 4 weeks; and the chemotherapy comprises mFOLFOX and is administered on days 1 and 15 of a 28-day cycle and then the cycle is repeated every 4 weeks. In some aspects, the oleclumab or antigen binding fragment is administered to the subject at a dose of 750 mg. In some aspects, the oleclumab or antigen binding fragment is administered to the subject at a dose of 1500 mg. In some aspects, the oleclumab or antigen binding fragment is administered to the subject at a dose of 2250 mg. In some aspects, the oleclumab or antigen binding fragment is administered to the subject at a dose of 3000 mg. In some aspects, the subject receiving the mFOLFOX is previously untreated or has progressed following gemcitabine-based chemotherapy. In some aspects the subject has not been exposed to 5-FU, capecitabine, or oxaliplatin.

In some aspects of the methods of inhibiting the growth of a tumor in a human subject comprising administering (i) oleclumab or antigen binding fragment thereof described herein, at least about 30% to about 70% of cells in a tumor sample obtained from the human subject have a staining intensity of at least 2.

In some aspects, provided herein are methods of inhibiting the growth of a tumor in a human subject comprising administering (i) oleclumab or antigen binding fragment thereof, (ii) durvalumab or antigen binding fragment thereof, and (iii) chemotherapy to the subject, wherein at least about 50% to about 90% of cells in a tumor sample obtained from the human subject have a staining intensity of 1, 2, or 3, as determined by IHC, and the oleclumab or antigen binding fragment is administered to the subject at a dose of 750 mg to 3000 mg every 2 weeks for four doses and then every 4 weeks; the durvalumab or antigen binding fragment thereof is administered to the subject at a dose of 1500 mg every four weeks; and the chemotherapy comprises mFOLFOX administered on days 1, and 15 of a 28-day cycle and then the cycle is repeated every 4 weeks. In some aspects, the oleclumab or antigen binding fragment is administered to the subject at a dose of 750 mg. In some aspects, the oleclumab or antigen binding fragment is administered to the subject at a dose of 1500 mg. In some aspects, the oleclumab or antigen binding fragment is administered to the subject at a dose of 2250 mg. In some aspects, the oleclumab or antigen binding fragment is administered to the subject at a dose of 3000 mg. In some aspects, the subject receiving the mFOLFOX is previously untreated or has progressed following gemcitabine-based chemotherapy. In some aspects the subject has not been exposed to 5-FU, capecitabine, or oxaliplatin.

In some aspects of the methods of inhibiting the growth of a tumor in a human subject comprising administering (i) oleclumab or antigen binding fragment thereof described herein, at least about 30% to about 70% of cells in a tumor sample obtained from the human subject have a staining intensity of at least 2.

In some aspects, provided herein are methods of inhibiting the growth of a tumor in a human subject comprising administering (i) oleclumab or antigen binding fragment thereof and (ii) chemotherapy to the subject, wherein at least about 50% to about 90% of cells in a tumor sample obtained from the human subject have a staining intensity of 1, 2, or 3, as determined by IHC; and wherein the oleclumab or antigen binding fragment is administered to the subject at a dose of 750 mg to 3000 mg every 2 weeks for four doses and then every 4 weeks; and the chemotherapy comprises FOLFIRINOX and is administered on day 1 of a 14-day cycle and then every 2 weeks. In some aspects, the oleclumab or antigen binding fragment is administered to the subject at a dose of 750 mg. In some aspects, the oleclumab or antigen binding fragment is administered to the subject at a dose of 1500 mg. In some aspects, the oleclumab or antigen binding fragment is administered to the subject at a dose of 2250 mg. In some aspects, the oleclumab or antigen binding fragment is administered to the subject at a dose of 3000 mg. In some aspects, the subject receiving the FOLFIRINOX is previously untreated or has progressed following gemcitabine-based chemotherapy. In some aspects the subject has not been exposed to 5-FU, capecitabine, or oxaliplatin.

In some aspects of the methods of inhibiting the growth of a tumor in a human subject comprising administering (i) oleclumab or antigen binding fragment thereof described herein, at least about 30% to about 70% of cells in a tumor sample obtained from the human subject have a staining intensity of at least 2.

In some aspects, provided herein are methods of inhibiting the growth of a tumor in a human subject comprising administering (i) oleclumab or antigen binding fragment thereof, (ii) durvalumab or antigen binding fragment thereof, and (iii) chemotherapy to the subject, wherein at least about 50% to about 90% of cells in a tumor sample obtained from the human subject have a staining intensity of 1, 2, or 3, as determined by IHC, and the oleclumab or antigen binding fragment is administered to the subject at a dose of 750 mg to 3000 mg every 2 weeks for four doses and then every 4 weeks; the durvalumab or antigen binding fragment thereof is administered to the subject at a dose of 1500 mg every four weeks; and the chemotherapy comprises FOLFIRINOX administered on day 1 of a 14-day cycle and then every 2 weeks. In some aspects, the oleclumab or antigen binding fragment is administered to the subject at a dose of 750 mg. In some aspects, the oleclumab or antigen binding fragment is administered to the subject at a dose of 1500 mg. In some aspects, the oleclumab or antigen binding fragment is administered to the subject at a dose of 2250 mg. In some aspects, the oleclumab or antigen binding fragment is administered to the subject at a dose of 3000 mg. In some aspects, the subject receiving the FOLFIRINOX is previously untreated or has progressed following gemcitabine-based chemotherapy. In some aspects the subject has not been exposed to 5-FU, capecitabine, or oxaliplatin.

In some aspects of the methods of inhibiting the growth of a tumor in a human subject comprising administering (i) oleclumab or antigen binding fragment thereof described herein, at least about 30% to about 70% of cells in a tumor sample obtained from the human subject have a staining intensity of at least 2.

In some aspects of the methods disclosed herein, the tumor is a 1^{st} line metastatic pancreatic ductal adenocarcinoma. In some aspects of the methods disclosed herein, the tumor is a 2^{nd} line metastatic pancreatic ductal adenocarcinoma.

In some aspects of the methods of inhibiting the growth of a tumor in a human subject disclosed herein, a CD73 IHC expression score of 2+ has been assigned to at least about 50% of the cells in a tumor sample from the subject. In some aspects of the methods of inhibiting the growth of a tumor in a human subject disclosed herein, a CD73 IHC expression score of 3+ has been assigned to at least about 50% of the cells in a tumor sample from the subject.

The CD73 IHC expression score has been detected by the IHC method described elsewhere herein.

In some aspects, the invention includes a method for extending an overall survival period in a human patient afflicted with metastatic pancreatic ductal adenocarcinoma (PDAC) comprising administering to the patient an immunotherapy disclosed herein, wherein the patient demonstrates progression-free survival for over 12 months. In some aspects, the progression-free survival of the patient can be extended, after the administration, for over about 13 months, about 14 months, about 15 months, about 16 months, about 17 months, about 18 months, about 2 years, about 3 years, about 4 years, about 5 years, about 6 years, about 7 years, about 8 years, about 9 years, or about 10 years as compared to standard of care therapy.

In some aspects, the invention includes a method for extending the overall response rate (ORR) that is at least about 10%, 15%, 20%, 30%, 40%, 50%, 60%, 70%, 75% longer or higher as compared to standard of care therapy.

In some aspects, the invention includes a method for extending the overall survival that is at least about 10%, 15%, 20%, 30%, 40%, 50%, 60%, 70%, 75% longer or higher as compared to standard of care therapy. In some aspects, the overall survival for a patient treated with a method of the invention is at least about 12, 13, 14, 15, 16, 17, 18, 19, 20, 21, 22, 23, 24 or more months.

In still other aspects, the invention is includes a method for reducing a tumor size at least by 10% in a human patient afflicted with metastatic pancreatic ductal adenocarcinoma (PDAC) comprising administering an immunotherapy disclosed herein, wherein the administration reduces the tumor size at least about 10%, about 20%, about 30%, about 40%, about 50%, about 60%, about 70%, about 80%, about 90%, or 100% compared to the tumor size prior to the administration. In some aspects, the method comprises identifying the patient as having a CD73 positive tumor prior to the administration.

In some aspects, the invention includes a method for increasing an objective response rate to be higher than 15% in a patient population. In some aspects, objective response rate is higher than 10%, 15%, 20%, 25%, 30%, 35%, 40%, 45%, 50%, 55%, 60%, 65%, 70%, 75% or higher. In some aspects, the method comprises identifying the patient as having a CD73 positive tumor prior to the administration.

In some aspects, each patient in the methods experiences (i) overall survival for over 12 months, (ii) tumor size reduction at least about 10%, about 20%, about 30%, about 40%, or about 50% compared to the tumor size prior to the administration, or (iii) both.

The methods of the invention, as a result of the administration of an immunotherapy disclosed herein, can treat the metastatic pancreatic ductal adenocarcinoma (PDAC), reduce the tumor size, inhibit growth of the tumor, eliminate the tumor from the patient, prevent a relapse of a tumor, induce a remission in a patient, or any combination thereof. In certain aspects, the administration of an immunotherapy disclosed herein induces a complete response. In other aspects, the administration of the immunotherapy disclosed herein induces a partial response.

In some aspects, the CD73 positive tumor comprises at least about 1%, at least about 2%, at least about 3%, at least about 4%, at least about 5%, at least about 7%, at least about 10%, at least about 15%, at least about 20%, at least about 25%, at least about 30%, at least about 40%, at least about 50%, at least about 60%, at least about 70%, at least about 80%, at least about 90%, or 100% cells expressing CD73.

In some aspects, CD73 expression is determined by receiving the results of an assay capable of determining CD73 expression.

In some aspects, a method of the invention alters the frequency of activated/proliferating and effector T cells. In some aspects, the T cells are measured by flow cytometry-based assays or immunohistochemistry.

In some aspects, a method of the invention alters protein or gene expression of biomarkers such as PD-1, PD-L1, CTLA-4, CD8, and IFN-γ.

In order to assess gene or protein expression (for example, CD73), in one aspect, a test tissue sample is obtained from the patient who is in need of the therapy. In some aspects, a test tissue sample includes, but is not limited to, any clinically relevant tissue sample, such as a tumor biopsy, a core biopsy tissue sample, a fine needle aspirate, or a sample of bodily fluid, such as blood, plasma, serum, lymph, ascites fluid, cystic fluid, or urine. In some aspects, the test tissue sample is from a primary tumor. In some aspects, the test tissue sample is from a metastasis. In some aspects, test tissue samples are taken from a subject at multiple time points, for example, before treatment, during treatment, and/or after treatment. In some aspects, test tissue samples are taken from different locations in the subject, for example, a sample from a primary tumor and a sample from a metastasis in a distant location.

In some aspects, the test tissue sample is a paraffin-embedded fixed tissue sample. In some aspects, the test tissue sample is a formalin-fixed paraffin embedded (FFPE) tissue sample. In some aspects, the test tissue sample is a fresh tissue (e.g., tumor) sample. In some aspects, the test tissue sample is a frozen tissue sample. In some aspects, the test tissue sample is a fresh frozen (FF) tissue (e.g., tumor) sample. In some aspects, the test tissue sample is a cell isolated from a fluid. In some aspects, the test tissue sample comprises circulating tumor cells (CTCs). In some aspects, the test tissue sample comprises tumor-infiltrating lymphocytes (TILs). In some aspects, the test tissue sample comprises tumor cells and tumor-infiltrating lymphocytes (TILs). In some aspects, the test tissue sample comprises circulating lymphocytes. In some aspects, the test tissue sample is an archival tissue sample. In some aspects, the test tissue sample is an archival tissue sample with known diagnosis, treatment, and/or outcome history. In some aspects, the sample is a block of tissue. In some aspects, the test tissue sample is dispersed cells. In some aspects, the sample size is from about 1 cell to about 1×10⁶ cells or more. In some aspects, the sample size is about 1 cell to about 1×10⁵ cells. In some aspects, the sample size is about 1 cell to about 10,000 cells. In some aspects, the sample size is about 1 cell to about 1,000 cells. In some aspects, the sample size is about 1 cells to about 100 cells. In some aspects, the sample size is about 1 cell to about 10 cells. In some aspects, the sample size is a single cell.

In another aspect, the assessment of expression can be achieved without obtaining a test tissue sample. In some aspects, selecting a suitable patient includes (i) optionally providing a test tissue sample obtained from a patient with cancer of the tissue, the test tissue sample comprising tumor cells and/or tumor-infiltrating inflammatory cells; and (ii) assessing the proportion of cells in the test tissue sample that express the gene/protein of interest, based on an assessment that the proportion of cells in the test tissue sample is higher than a predetermined threshold level.

### 7.5 Patient Populations

Provided herein are clinical methods for treating cancers (e.g., metastatic pancreatic ductal adenocarcinoma (PDAC) in human patients using any method disclosed herein, for example, an ant-CD73 antibody or antigen-binding fragment thereof, e.g., oleclumab or antigen binding fragment thereof, i.e., (for example, MEDI9447) or antigen-binding fragment thereof, administered as a single agent or optionally in combination with one or more chemotherapeutic agents.

In some aspects, the subject is human. In some aspects, the human subject is an adult ≥18 years of age with histologically or cytologically confirmed pancreatic adenocarcinoma. In some aspects, the subject has previously untreated metastatic PDAC (1L metastatic PDAC).

In some aspects, the cells in a tumor sample obtained from the human subject express CD73. In some aspects a tumor sample obtained from the human subject express high CD73 expression. Tumor samples from patients considered to have High CD73 expression have a P-score ranging from at least about 50% to about 90%. Tumor samples from patients considered to have High CD73 expression have a 2+3+P-score ranging from at least about 30% to about 70%.

In some aspects, at least about 50% to about 90% of cells in a tumor sample obtained from the human subject have a staining intensity of 1, 2, or 3, as determined by IHC.

In some aspects, at least about 30% to about 70% of cells in a tumor sample obtained from the human subject have a staining intensity of at least 2.

In some aspects, at least about 30% to about 70% of cells in a tumor sample obtained from the human subject have a staining intensity of at least 3.

### 7.6 Outcomes

A patient treated according to the methods disclosed herein preferably experience improvement in at least one sign of cancer. In some aspects, improvement is measured by evaluation of plasma and serum levels of soluble factors such as soluble CD73 and soluble CD73 enzymatic activity. In some aspects, improvement is measured by evaluation of immune mediators of antitumor immune response, which measurement can be used to explore their association with treatment and clinical outcome. In some aspects, improvement is measured by investigation of prognostic markers of PDAC such as CD73 expression, microsatellite stable (MSS) status and tumor mutational burden for association with disease response. In some aspects, tumor response to the administration of the anti-CD73 antibody or antigen binding fragment thereof can be determined by Investigator review of tumor assessments and defined by the RECIST v1.1 guidelines. Additional tumor measurements can be performed at the discretion of the Investigator or according to institutional practice.

In some aspects, improvement is measured by the evaluation of tumor samples for mutational burden. In some aspects, improvement is measured by CD73 protein expression level. In some aspects, improvement is measured by the evaluation of the immunosuppressive biomarker PD-L1 protein. In some aspects, improvement is measured by the evaluation of immune markers that can include but are not be limited to PD-1, CD3, CD4, CD8, forkhead box P3, and granzyme B by IHC analysis to evaluate baseline expression in 1L and 2L PDAC.

CD73 expression is determined in a College of American Pathologists (CAP)/Clinical Laboratory Improvement Amendments (CLIA) laboratory using a validated, fully automated, IHC assay.

In some aspects, the primary efficacy endpoint is OR, which is defined as best overall response of confirmed complete response (CR) or confirmed partial response (PR) according to RECIST version 1.1. The best overall response is defined as the best response (in the order of CR (complete response), PR, (stable disease) SD, progressive disease (PD), and NE (not evaluable) among all overall responses recorded from the start of treatment until objective documentation of PD, or the last evaluable disease assessment in the absence of PD prior to the initiation of subsequent anticancer therapy or end of the study, whichever occurs first. The best overall response of CR or PR must be confirmed, which means a response of CR/PR is recorded at a visit and confirmed by repeat imaging not less than 28 days (4 weeks) after the visit when the response was first observed with no evidence of progression between the initial and CR/PR confirmation visit. The objective response rate (ORR) can be estimated by the proportion of OR, and its 95% confidence interval (CI) is estimated using the exact binomial distribution. Comparison of arms for ORR is obtained from Cochran-Mantel-Haenszel test.

In another aspect, the patient treated experiences tumor shrinkage and/or decrease in growth rate, i.e., suppression of tumor growth. In some aspects, unwanted cell proliferation is reduced or inhibited. In some aspects, one or more of the following can occur: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent.

In other aspects, administration of an antibody or antigen-binding fragment thereof according to any of the methods provided herein produces at least one therapeutic effect selected from the group consisting of reduction in size of a tumor, reduction in number of metastatic lesions appearing over time, complete remission, partial remission, or stable disease.

In some aspects, one or more tumor assessments can be used to determine tumor response to administration of an anti-CD73 antibody or antigen binding fragment thereof according to any of the methods provided herein. Tumor assessments can include the following evaluations: cross-sectional imaging using CT or magnetic resonance imaging (MRI) scan of the chest, abdomen, pelvis; and brain. CT or MRI scan of the chest, abdomen, and pelvis will be performed with each disease assessment for all subjects. Additionally, CT or MRI scan of the brain will be performed at screening for all subjects with clinical concern for brain metastasis. Any subjects with brain metastases at screening or any subjects who develop neurologic or other clinical symptoms that warrant imaging must also have brain imaging with each disease assessment. The preferred method of disease assessment is CT with contrast; if CT with contrast is contraindicated, CT without contrast is preferred over MRI. The preferred method for CNS imaging is MRI; if CT scan is performed, CT with contrast is required. The same method is preferred for all subsequent tumor assessments.

In some aspects, the sample is a formalin-fixed paraffin embedded (FFPE) sample. In some aspects, the sample is a fresh sample. Tumor samples (e.g., biopsies) can be used to identify predictive and/or pharmacodynamic biomarkers associated with immune and tumor microenvironment. Such biomarkers can be determined from assays including IHC, tumor mutation analysis, RNA analysis, and proteomic analyses. In certain aspects, expression of tumor biomarkers are detected by RT-PCR, in situ hybridization, RNase protection, RT-PCR-based assay, immunohistochemistry, enzyme linked immunosorbent assay, in vivo imaging, or flow cytometry. In certain aspects, expression of tumor biomarkers are detected by the CD73 immunohistochemistry (IHC) assay described elsewhere herein.

### 7.7 Pharmaceutical Compositions

Pharmaceutical compositions suitable for administration to human patients are typically formulated for parenteral administration, e.g., in a liquid carrier, or suitable for reconstitution into liquid solution or suspension for intravenous administration.

In general, such compositions typically comprise a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions may be employed as carriers, particularly for injectable solutions. Liquid compositions for parenteral administration can be formulated for administration by injection or continuous infusion. Routes of administration by injection or infusion include intravenous, intraperitoneal, intramuscular, intrathecal and subcutaneous.

The following examples are offered by way of illustration and not by way of limitation.

### 8. EXAMPLES

The example in this Section (i.e., Section 6) is offered by way of illustration, and not by way of limitation.

### 8.1 Example 1: Oleclumab Treatment with or without Durvalumab in Combination with Chemotherapy in Subjects with Metastatic Pancreatic Ductal Adenocarcinoma

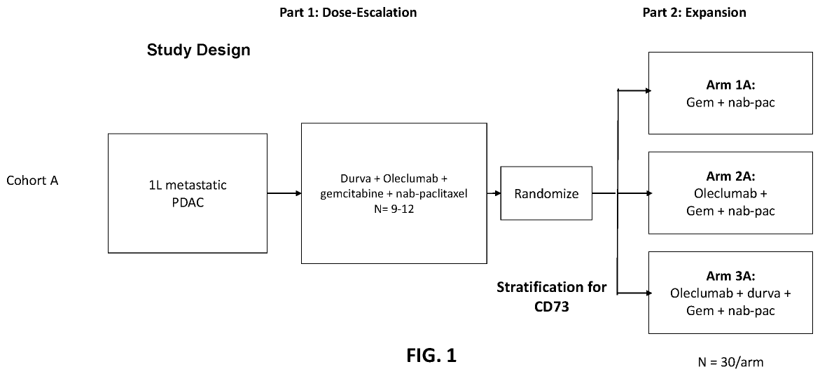

This study is a Phase 1b/2, multicenter, open-label, dose-escalation and dose-expansion study to assess the safety, preliminary antitumor activity, immunogenicity, and PK of oleclumab with or without durvalumab in combination with chemotherapy administered in subjects with metastatic PDAC. Subjects with previously untreated metastatic PDAC (1L metastatic PDAC) were enrolled in Cohort A. (FIG. 1) The study includes 2 parts, dose escalation (Part 1) and dose expansion (Part 2). The target population for the study is subjects ≥18 years of age diagnosed with histologically or cytologically confirmed pancreatic adenocarcinoma. Subjects with previously untreated metastatic PDAC (1L metastatic PDAC) were enrolled in Cohort A.

Treatment Groups and Regimens

Up to approximately 339 subjects are enrolled in this study with approximately 24 subjects in Part 1 (dose escalation) and approximately 315 subjects in Part 2 (dose expansion). All subjects in both cohorts are treated until disease progression (and the treatment criteria in the setting of progressive disease are not met), intolerable toxicity, withdrawal of subject consent, or another discontinuation criterion is met.

Part 1 (Dose Escalation)

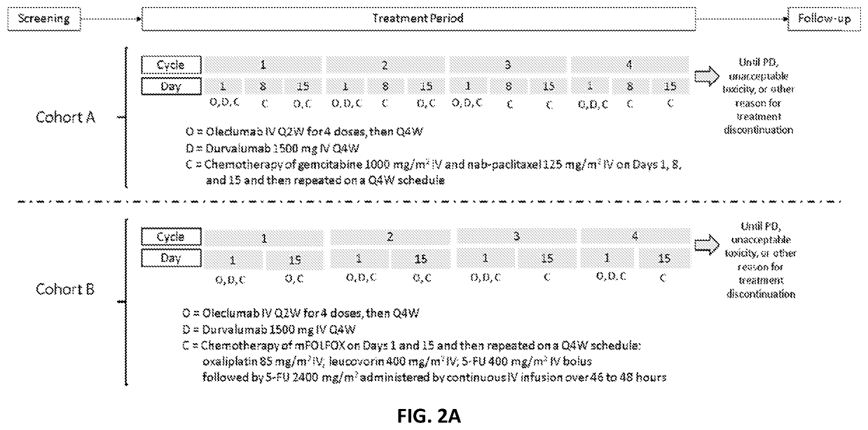

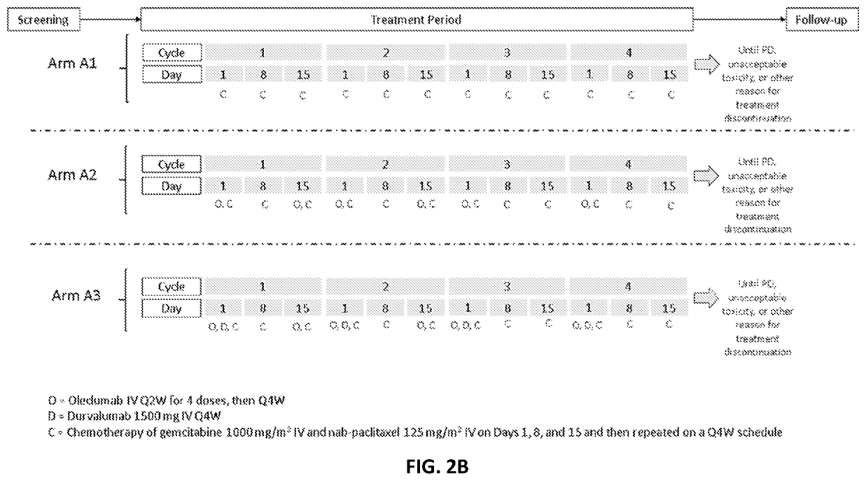

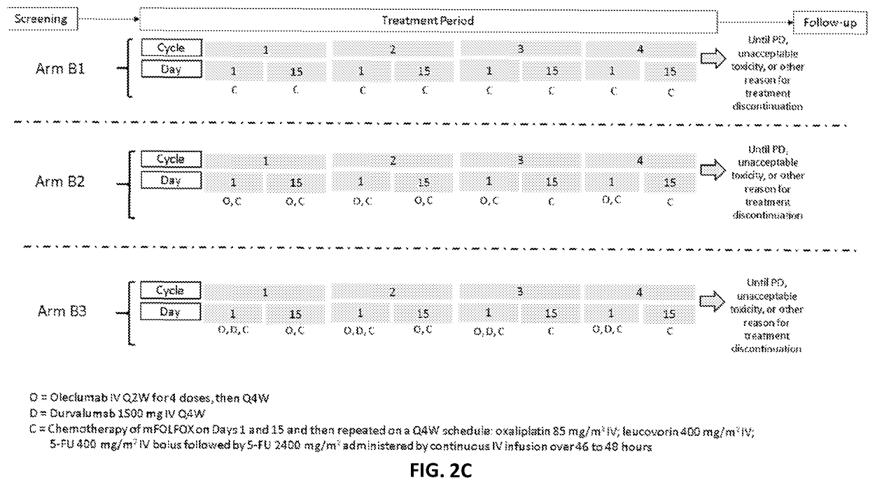

Up to approximately 24 subjects are enrolled in Part 1 (dose escalation) (FIG. 2A). From 9 to approximately 12 subjects with 1L metastatic PDAC are enrolled in Cohort A. From 9 to approximately 12 subjects with 2L metastatic PDAC are enrolled in Cohort B. A single dose level for durvalumab and chemotherapy (gemcitabine/nab-paclitaxel for Cohort A; mFOLOX for Cohort B) are used in combination with oleclumab as detailed below.
Oleclumab at one of 3 dose levels:
   Dose level −1 (3-6 subjects): 750 mg IV every 2 weeks (Q2W) for 4 doses, then every 4 weeks (Q4W); if the MTD is exceeded at the starting dose level (Dose Level 1)
Dose level 1 (3-6 subjects):1500 mg IV Q2W for 4 doses, then Q4W (starting dose level)
   Dose level 2 (6 subjects): 3000 mg IV Q2W for 4 doses, then Q4W (highest planned dose level)
Durvalumab 1500 mg IV Q4W
Cohort A: Gemcitabine 1000 mg/m2 IV and nab-paclitaxel 125 mg/m2 IV on Days 1, 8, and 15 and then repeated on a Q4W schedule
Cohort B: mFOLFOX on Days 1 and 15 and then repeated on a Q4W schedule: oxaliplatin 85 mg/m2 IV; leucovorin 400 mg/m2 IV; 5-FU 400 mg/m2 IV bolus followed by 5-FU 2400 mg/m2 administered by continuous IV infusion over 46 to 48 hours

Part 2 (Dose Expansion)

Up to approximately 315 subjects are enrolled in Part 2 (dose expansion), with up to approximately 210 subjects in Cohort A. Subjects are stratified by CD73 expression level and randomized 1:1:1 to one of 3 treatment arms per cohort (approximately 70 subjects per treatment arm in Cohort A). Expression level is defined as follows: CD73 high=tumor samples that have CD73 expression of 2+ or 3+ intensity in ≥50% of tumor cells. CD73 low=tumor samples with no CD73 expression in tumor cells or <50% of tumor cells with 2+ or 3+ intensity. The dose level for oleclumab is determined during Part 1 (dose escalation). The dose and treatment regimens for durvalumab and chemotherapy are the same as in dose escalation. The treatment arms for Cohort A and Cohort B are detailed below and in FIGS. 2B and 2C:

Cohort A

Arm A1
Gemcitabine 1000 mg/m2 IV and nab-paclitaxel 125 mg/m² IV on Days 1, 8, and 15 and then repeated on a Q4W schedule

Arm A2
Oleclumab IV Q2W for 4 doses, then Q4W, and
Gemcitabine 1000 mg/m2 IV and nab-paclitaxel 125 mg/m2 IV on Days 1, 8, and 15 and then repeated on a Q4W schedule

Arm A3
Oleclumab IV Q2W for 4 doses, then Q4W, and
Durvalumab 1500 mg IV Q4W, and
Gemcitabine 1000 mg/m2 IV and nab-paclitaxel 125 mg/m² IV on Days 1, 8, and 15 and then repeated on a Q4W schedule

Cohort B
Arm B1 (35 subjects): mFOLFOX
Arm B2 (35 subjects): oleclumab and mFOLFOX, and
Arm B3 (35 subjects): oleclumab, durvalumab, and mFOLFOX

Efficacy

The efficacy analyses of antitumor activity is based on the intent-to-treat (ITT) population (defined as all subjects who are randomized and receive any amount of investigational product, analyzed according to randomized treatment assignment). The rates of OR and DC based on RECIST v1.1 are summarized with 95% confidence interval based on the exact binomial distribution. Time-to-event endpoints (DoR, PFS, and OS) are analyzed using the Kaplan-Meier method.

Immunogenicity and Pharmacokinetics

Only subjects who received at least 1 dose of oleclumab and/or durvalumab and provide at least 1 post-treatment sample are evaluated.

For each cohort, the immunogenic potential of combinations is assessed by summarizing the number and percentage of subjects who develop detectable anti-drug antibodies (ADAs). Individual oleclumab and durvalumab concentrations are tabulated by dose cohort along with descriptive statistics. Non-compartmental PK data analysis is performed from each dose cohort with scheduled PK sample collection where data allow. Relevant descriptive statistics of non-compartmental PK parameters can include: area under concentration-time curve, maximum observed concentration (Cmax), time to reach Cmax, clearance, volume of distribution, and terminal half-life.

Pharmacodynamics of oleclumab are assessed by changes in gene expression in whole blood and soluble factors including, but not limited to, soluble CD73, cytokines, and ctDNA tabulated by cohort. Descriptive statistics are used to describe subject and cohort specific changes. Tissue obtained as part of screening procedure and optional biopsies obtained at the end of treatment are assessed for establishing CD73 expression by IHC. Based on availability of tissue, a panel of additional, immune-relevant markers expressed on tumor-infiltrating lymphocytes or on tumor cells is assessed. Other tissue-based approaches are pursued, including gene expression methods (e.g., for detection of, but not limited to, interferon-gamma [IFN-γ] signaling genes such as CXCL9, CXCL10, and IFN-γ itself), and/or somatic mutation detection methodologies.

### Objectives and Study Endpoints

Primary Objectives and Endpoints:

Part 1 (Dose Escalation)
To assess the safety and tolerability of oleclumab plus durvalumab in combination with chemotherapy administered in subjects with metastatic PDAC
Dose-limiting toxicities (DLTs), incidence of adverse events (AEs) and serious adverse events (SAEs), and clinically meaningful changes from baseline in clinical laboratory parameters, vital signs, and electrocardiogram (ECG) results

Part 2 (Dose Expansion)
To evaluate the preliminary antitumor activity of oleclumab with or without durvalumab in combination with gemcitabine and nab-paclitaxel compared to gemcitabine and nab-paclitaxel administered in subjects with first-line (1L) metastatic PDAC
Objective response (OR) according to Response Evaluation Criteria in Solid Tumors (RECIST) version 1.1 (v1.1)
To evaluate the preliminary antitumor activity of oleclumab with or without durvalumab in combination with chemotherapy in subjects with second-line (2L) metastatic PDAC OR according to RECIST v1.1

Secondary Objectives and Endpoints:

Part 1 (Dose Escalation)
To evaluate the preliminary antitumor activity of oleclumab plus durvalumab in combination with gemcitabine and nab-paclitaxel administered in subjects with 1L metastatic PDAC OR and disease control (DC) according to RECIST v1.1

Part 2 (Dose Expansion)
To assess the safety and tolerability of oleclumab with or without durvalumab in combination with chemotherapy administered in subjects with metastatic PDAC
Incidence of AEs and SAEs and clinically meaningful changes from baseline in clinical laboratory parameters, vital signs, and ECG results
To evaluate the preliminary antitumor activity of oleclumab with or without durvalumab in combination with gemcitabine and nab-paclitaxel compared to gemcitabine and nab-paclitaxel administered in subjects with 1L metastatic PDAC
   Overall survival (OS); progression-free survival (PFS), duration of response (DoR), and DC according to RECIST v1.1
To evaluate the preliminary antitumor activity of oleclumab with or without durvalumab in combination with chemotherapy compared to chemotherapy alone in the population defined by CD73 expression
   OS; OR and PFS according to RECIST v1.1 by CD73 expression at baseline

Part 1 (Dose Escalation) and Part 2 (Dose Expansion)
To assess the immunogenicity of oleclumab and durvalumab in combination with chemotherapy administered in subjects with metastatic PDAC
Development of detectable anti-drug antibodies (ADAs) following oleclumab and durvalumab
To determine the pharmacokinetic (PK) profile of oleclumab and durvalumab in combination with chemotherapy administered in subjects with metastatic PDAC
Summary PK for oleclumab, durvalumab, and selected chemotherapies and/or their metabolites

Dose-Limiting Toxicity (DLT)

DLTs are evaluated during Part 1 (dose escalation). The period for DLT-evaluation is from the first dose of all study treatments through Day 28. Subjects who do not complete the DLT-evaluation period or did not receive all planned doses of oleclumab, durvalumab, or chemotherapy during this time for reasons other than a DLT are considered non-evaluable for DLT assessment and are replaced with another subject at the same dose level, but will still be considered when reviewing toxicity from this cohort. Grading of DLTs is according to the National Cancer Institute Common Terminology Criteria for Adverse Events (NCI CTCAE version 4.03). A DLT is defined as any Grade 3 or higher toxicity or any of the events listed below that occurs during the DLT-evaluation period. Toxicity that is clearly and directly related to the primary disease, chemotherapy alone, or to another etiology will not be considered DLTs. The following will be DLTs:

Immune-Mediated Adverse Events (imAEs)
Any Grade 4 imAE (excluding asymptomatic lipase and/or amylase elevation)
Any ≥Grade 3 colitis
Any ≥Grade 3 nausea, vomiting, or diarrhea that does not resolve to Grade 2 or less within 3 days of the initiation of maximal supportive care
Any ≥Grade 3 pneumonitis or ILD
Any Grade 2 pneumonitis or ILD for which the symptomatology does not resolve within 7 days of the initiation of maximal supportive care

Anemia
Grade 4 anemia of any duration
Grade 3 anemia if associated with clinical sequelae or requires transfusion of >2 units of red blood cells

Thrombocytopenia
Grade 4 thrombocytopenia ≥7 days
Grade 3 or 4 thrombocytopenia, regardless of duration, associated with Grade 3 or higher hemorrhage

Neutropenia and/or Febrile Neutropenia
Grade 4 febrile neutropenia of any duration
Grade 3 febrile neutropenia lasting ≥5 days while receiving maximal supportive care
Grade 4 neutropenia lasting >7 days

Liver Function Tests
Isolated Grade 3 liver transaminase elevation or isolated Grade 3 total bilirubin (TBL) that does not downgrade to Grade 1 or less within 14 days after onset with optimal medical management, including systemic corticosteroids.
Isolated Grade 4 liver transaminase elevation or TBL regardless of duration.
Any increase in AST or ALT >3×upper limit normal (ULN) and concurrent increase in TBL >2×ULN (Hy's Law) without evidence of cholestasis or alternative explanations (eg, viral hepatitis, disease progression in the liver)

Or any other toxicity that is greater than that at baseline, is clinically significant and/or unacceptable and is judged to be a DLT by the DEC

A DLT Excludes the Following:
Grade 3 endocrine disorder (thyroid, pituitary, and/or adrenal insufficiency) that is managed with or without systemic corticosteroid therapy and/or hormone replacement therapy
Grade 3 inflammatory reaction attributed to a local antitumor response (eg, inflammatory reaction at sites of metastatic disease, lymph nodes, etc) that resolved to Grade 1 or less within 30 days
Concurrent vitiligo or alopecia of any AE grade
Isolated laboratory changes of any grade without clinical sequelae or clinical significance that are not defined as a DLT above
   Immune-mediated AEs are defined as AEs of an immune nature (ie, inflammatory) in the absence of a clear alternative etiology. In the absence of clinical abnormality, repeat laboratory testing will be conducted to confirm significant laboratory findings prior to designation as a DLT.

Inclusion Criteria
1. Age ≥18 years at the time of screening or age of consent according to law.
2. Written informed consent and any locally required authorization (eg, data privacy) obtained from the subject prior to performing any protocol-related procedures, including screening evaluations.
3. Eastern Cooperative Oncology Group (ECOG) performance status of 0 or 1.
4. Weight ≥35 kg.
5. Subjects diagnosed with histologically or cytologically confirmed pancreatic adenocarcinoma:

For Cohort A: Subjects must not have received systemic therapy for metastatic pancreatic adenocarcinoma. If subjects received prior neoadjuvant or adjuvant chemotherapy and progressed within 6 months of the last dose, then this should be considered as a prior line of systemic therapy.
6. Subjects must have at least 1 measurable lesion according to RECIST version 1.1.
(a) A previously irradiated lesion can be considered a target lesion if the lesion is well defined, measurable per RECIST, and has clearly progressed.
(b) Subjects must have a non-target lesion that can be biopsied at acceptable risk (if biopsy is required for enrollment) as judged by the investigator, or if no other lesion is suitable for biopsy, then a RECIST target lesion used for biopsy must be ≥2 cm in longest diameter
7. All subjects must consent to providing archival tumor specimens (core biopsies or larger resection, no fine-needle aspiration samples) for correlative biomarker studies and CD73 expression testing for stratification if tumor tissue is available. If archival specimen (≤12 months old) is not available, subjects must consent to a fresh biopsy.
8. Adequate organ and marrow function as defined in Table 5 (4.1.2-1).

Exclusion Criteria
1. Receipt of any conventional or investigational anticancer therapy within 21 days or palliative radiotherapy within 14 days prior to the scheduled first dose of study treatment.
2. Prior receipt of any immune-mediated therapy including, but not limited to, other anti-CTLA-4, anti-PD-1, anti-PD-L1 antibodies and agents targeting CD73, CD39, or adenosine receptors, excluding therapeutic anticancer vaccines.
3. Concurrent enrollment in another therapeutic clinical study. Enrollment in observation studies will be allowed.
4. Any toxicity (excluding alopecia) from prior standard therapy that has not been completely resolved to baseline at the time of consent. Subjects with NCI CTCAE version 4.03 Grade 1 or 2 toxicities that are deemed stable or irreversible can be enrolled on a case-by-case basis with prior consultation and agreement with the medical monitor.
5. Subjects with a history of venous thrombosis within the past 3 months prior to the scheduled first dose of study treatment. NOTE: Subjects with thrombosis due to mechanical obstruction by the tumor that is found incidentally and is asymptomatic and does not require therapy may be enrolled at the investigator's discretion and should be closely monitored.
6. Subjects with prior history of myocardial infarction, transient ischemic attack, or stroke within the past 3 months prior to the scheduled first dose of study treatment.
7. Active or prior documented autoimmune disorders within the past 3 years prior to the scheduled first dose of study treatment. The following are exceptions to this criterion:
   (a) Subjects with vitiligo or alopecia.
   (b) Subjects with hypothyroidism (eg, following Hashimoto syndrome) stable on hormone replacement or psoriasis not requiring systemic treatment.
   (c) Any chronic skin condition that does not require systemic therapy.
   (d) Subjects with celiac disease controlled by diet alone.
8. Subjects with confirmed human immunodeficiency virus (even if viral load is undetectable), chronic or active hepatitis B or C, or active hepatitis A.
9. History of primary immunodeficiency, solid organ transplantation, or active tuberculosis. In settings where there is a clinical or radiographic evidence of tuberculosis, active disease must be excluded prior to enrollment.
10. Other invasive malignancy within 2 years. Noninvasive malignancies (ie, cervical carcinoma in situ, in situ prostate cancer, non-melanomatous carcinoma of the skin, ductal carcinoma in situ of the breast that has been surgically cured) are excluded from this definition.
11. Known allergy or hypersensitivity to investigational product formulations.
12. Uncontrolled intercurrent illness including, but not limited to ongoing or active infection requiring antibiotic therapy, uncontrolled hypertension, bleeding diatheses, or psychiatric illness/social situations that would limit compliance with study requirements, substantially increase risk of incurring AEs, or compromise the ability of the subject to give written informed consent.
13. Any history of leptomeningeal disease or cord compression.
14. Untreated CNS metastatic disease. Note: Subjects previously treated for CNS metastases that are radiographically and neurologically stable for at least 28 days and do not require corticosteroids (of any dose) for CNS symptomatic management for at least 14 days prior to the scheduled first dose of study treatment will be eligible.
15. Current or prior use of immunosuppressive medication within 14 days prior to the scheduled first dose of study treatment. The following are exceptions to this criterion:
   (a) Intranasal, topical, inhaled corticosteroids or local steroid injections (eg, intra-articular injection).
   (b) Systemic corticosteroids at physiologic doses not to exceed 10 mg/day of prednisone or equivalent.
   (c) Steroids as premedication for hypersensitivity reactions (eg, computed tomography [CT] scan premedication).
16. Receipt of live, attenuated vaccine within 28 days prior to the scheduled first dose of study treatment (Note: Subjects, if enrolled, should not receive live vaccine during the study and 180 days after the last dose of study treatment). Vaccination with an inactivated vaccine is permitted at any time.
17. Major surgery (as defined by the investigator) within 28 days prior to scheduled first dose of study treatment or still recovering from prior surgery. Local procedures (eg, placement of a systemic port, core needle biopsy, and prostate biopsy) are allowed if completed at least 24 hours prior to the administration of the first dose of study treatment.
18. Females who are pregnant, lactating, or intend to become pregnant during their participation in the study.
19. Subjects who are involuntarily incarcerated or are unable to willingly provide consent or are unable to comply with the protocol procedures.
20. Any condition that, in the opinion of the investigator, would interfere with safe administration or evaluation of the investigational products or interpretation of subject safety or study results.
21. Known allergy or hypersensitivity to gemcitabine, nab-paclitaxel, oxaliplatin, leucovorin, or 5-FU.

### 8.2 Example 2: CD73 Immunohistochemistry Assay and Scoring Criteria

CD73 expression in tumor samples was evaluated by immunohistochemistry. Briefly, after de-waxing in xylenes and rehydration through a graded series of alcohol, Target Retrieval Solution, pH 6 (Dako, cat. no. K8005) is used for retrieval of the CD73 antigens/epitopes in the Dako PT link at 97° C. for 20 min. The primary antibody (Abcam/#ab124725 (clone EPR6115)) and all required isotype control antibodies were prepared fresh for each run using Antibody Diluent (Dako, cat. no. S0809). Target recognition for CD73 in FFPE sections uses the signal amplification/detection reagents from Dako Rabbit Envision+ kit (cat. no. K4011). All samples are counterstained using hematoxylin (Dako, cat. no. S3301). Storage and handling of all commercial reagents is in accordance with the manufacturers' instructions. The CD73 IHC protocol at the CAP/CLIA test facility was automated with the Dako Autostainer Link48.

Two methods of scoring were employed throughout the study. Both methods employed a direct estimation of the percentage of tumor cells staining at 0, 1, 2, and 3 intensities, with 0 indicating the lack of staining and 3 indicating strong staining.

Only tumor membrane staining was considered. Luminal staining was scored as a representation of the cell nest in which the lumen was located. For the first method, the H-score, the proportion of tumor cells staining at a certain intensity was multiplied by that intensity factor and each product at the four intensity levels are summed.

H-score=[(% at <1)*0]+[(% at 1)*1]+[(% at 2)*2]+[(% at 3)*3].

The second method of scoring used was the P-score,

Pathologists counted cell surface CD73 staining intensity at 0, 1, 2, and 3 intensities. The percentage of cells stained at 1, 2, and 3, and the percentage of cells stained at 2 and 3 within each sample were considered. CD73 expression was assessed using one of two different P scores: (1) the P-score, and (2) the 2+3+P-score. The equations for each score are provided below.

P-score=(% at 1)+(% at 2)+(% at 3). The P-score is the sum of the percentage of tumor cells staining at 1, 2, or 3 intensities.

2+3+P-score=(% at 2)+(% at 3). The 2+3+P-score is the sum of the percentage of tumor cells staining at 2, or 3 intensities.

Tumor samples from patients considered to have High CD73 expression had a P-score threshold of about 70%.

Tumor samples from patients considered to have High CD73 expression had a 2+3+P-score threshold of at least about 50%.

Patients were stratified by CD73 2+3+P-score and randomized 1:1:1 to one of 3 treatment arms.

### 8.3 Example 3: Results of the Phase Lb/2 Study

The median overall survival in months (mOS) with standard of care (chemotherapy only) for CD73 high patients is 7.9 months. This is worse than the 10.6 month mOS of all patients, which suggests that high CD73 is poor prognostic marker. The median overall survival with oleclumab, durvalumab, and chemotherapy is 12.1 months.

As noted above, in this Phase 1b/2 study, patients were stratified by CD73 2+3+P-score with patients ≥50% tumor cell 2+3+P-score in the high group and <50% tumor cell 2+3+ in the low group and randomized 1:1:1 to one of 3 treatment arms per cohort.

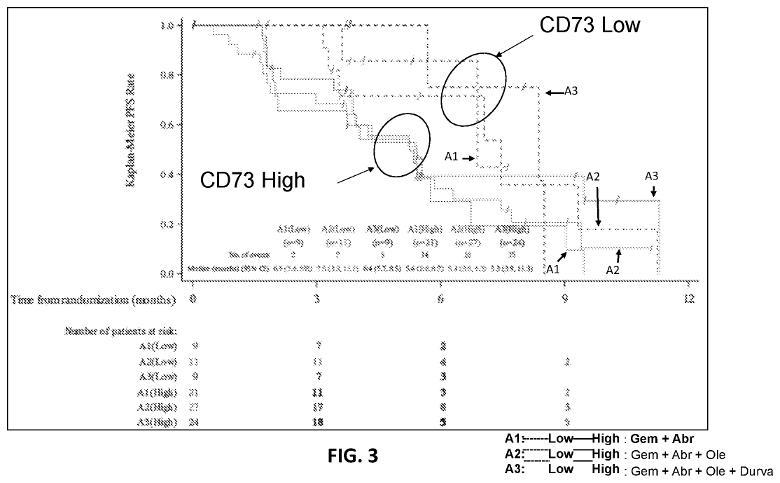

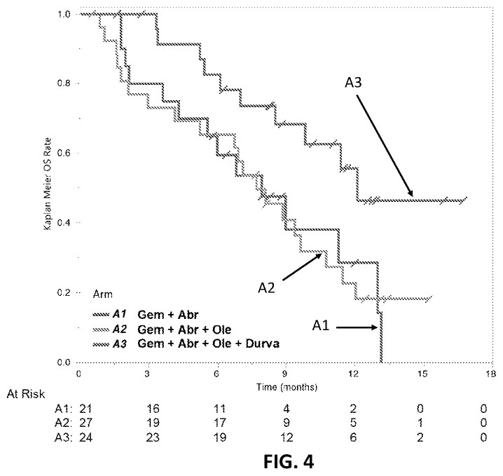

The high CD73 expression sub-group was associated with lower response to chemotherapy (gemcitabine/abraxane (nab-paclitaxel)). However, the addition of oleclumab+/−durvalumab to chemotherapy (gemcitabine/nab-paclitaxel) showed an increased overall response rate in the CD73 high sub-group. The high CD73 expression sub-group was associated with shorter progression-free survival than the low CD73 sub-group regardless of the addition of oleclumab+/−durvalumab to chemotherapy (gemcitabine/nab-paclitaxel). (Table 3 and FIGS. 3 and 4.)

TABLE 3
Enrichment of response in CD73 high population at a minimum of 16 weeks of follow up

| | CD73 Low | | | CD73 High (≥50% TC, 2+3+) | | |
|---|---|---|---|---|---|---|
| | Arm A1 | Arm A2 | Arm A3 | Arm A1 | Arm A2 | Arm A3 |
| | N = 7 | N = 10 | N = 8 | N = 19 | N = 25 | N = 22 |
| ORR, n (%) | 3 (42.9%) | 1 (10.0%) | 4 (50.0%) | 2 (10.5%) | 6 (24.0%) | 6 (27.3%) |
| CR | 0 | 0 | 0 | 0 | 0 | 0 |
| PR | 3 (42.9%) | 1 (10.0%) | 4 (50.0%) | 2 (10.5%) | 6 (25.0%) | 6 (27.3%) |
| SD | 4 (57.1%) | 9 (90.0%) | 4 (50.0%) | 12 (63.2%) | 12 (50.0%) | 11 (50.0%) |
| Unconfirmed PR | 2 (28.6%) | 3 (30.0%) | 0 | 2 (10.5%) | 2 (8.3%) | 3 (13.6%) |
| PD | 0 | 0 | 0 | 5 (26.3%) | 3 (12.5%) | 5 (22.7%) |
| NE | 0 | 0 | 0 | 0 | 4 (16.0%) | 0 |
| CR + PR (conf + | 5 (71.4%) | 4 (40.0%) | 4 (50.0%) | 4 (21.1%) | 8 (32.0%) | 9 (40.9%) |
| unconf) | | | | | | |

ORR = objective response rate
CR = complete response
PR = partial response
SD = stable disease
PD = progressive disease
NE = not evaluable

As shown, the overall response rate was higher in the CD73 High group of patients at a minimum of 16 weeks follow up in Arm A3 compared to Arm A1. CR and PR were evident in CD73 High patients with the following percentages: 21.1% (Arm A1), 32.0% (Arm A2), and 40.9% (Arm A3). This demonstrates that treatment of CD73 high patients with oleclumab and durvalumab in addition to chemotherapy (gemcitabine/nab-paclitaxel) can result in better response rate compared to chemotherapy alone.

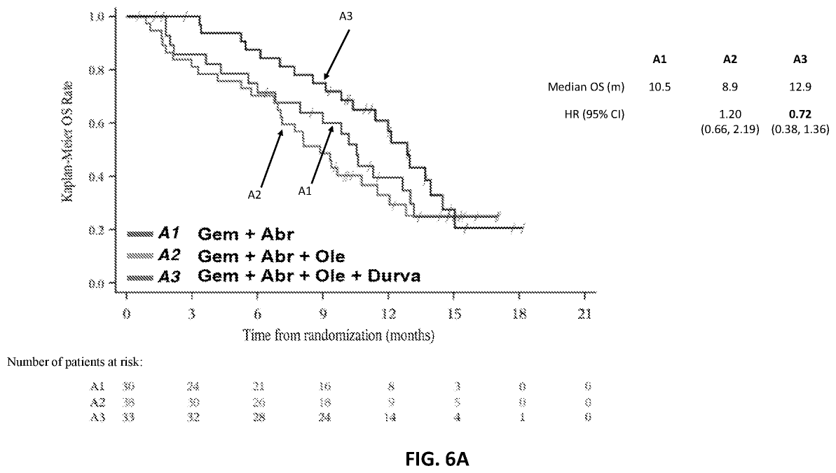

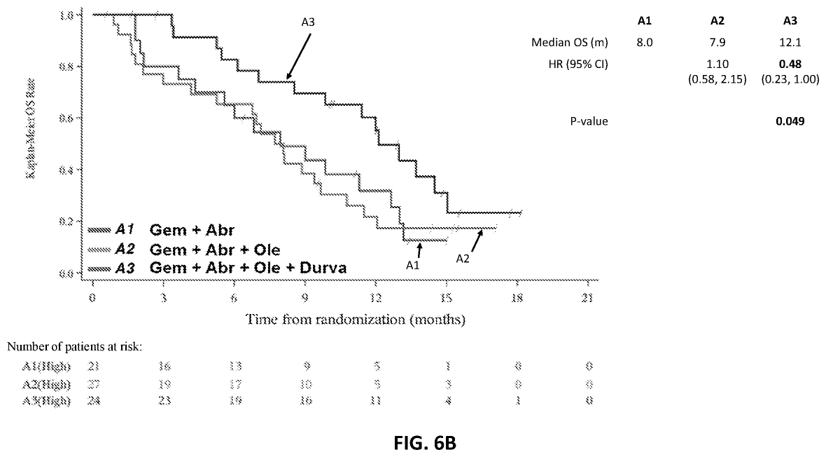

This example demonstrates that the addition of oleclumab+durvalumab to gemcitabine/nab-paclitaxel led to an overall survival benefit in the CD73 high patient group as displayed in FIG. 6B.

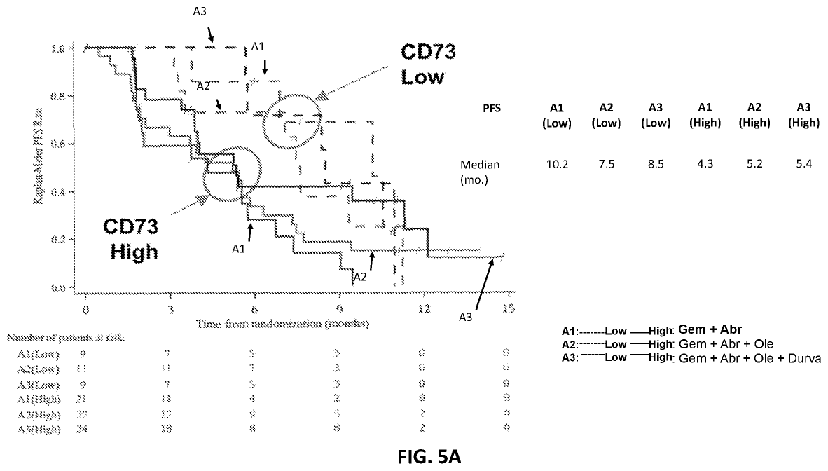

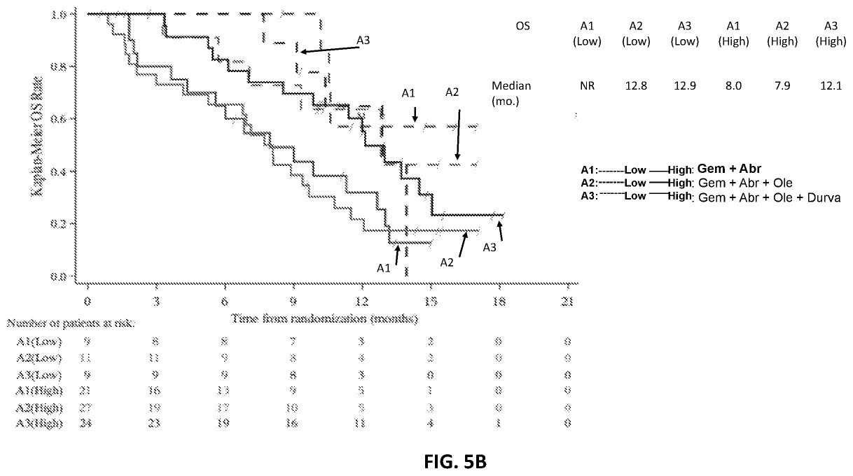

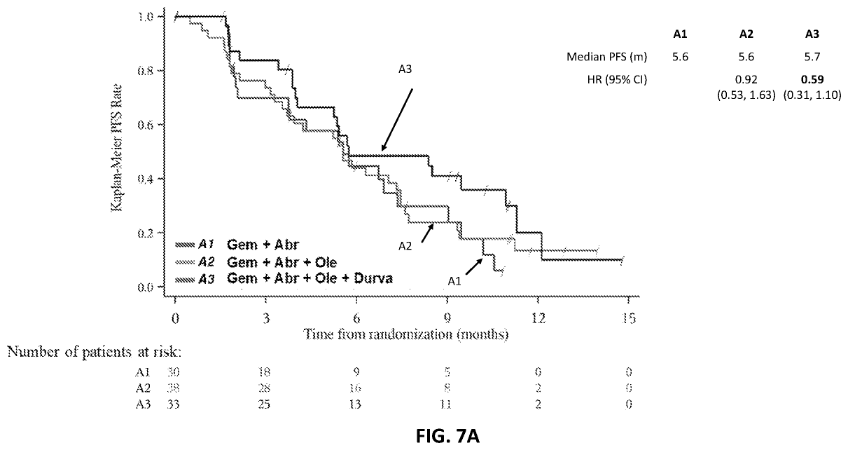

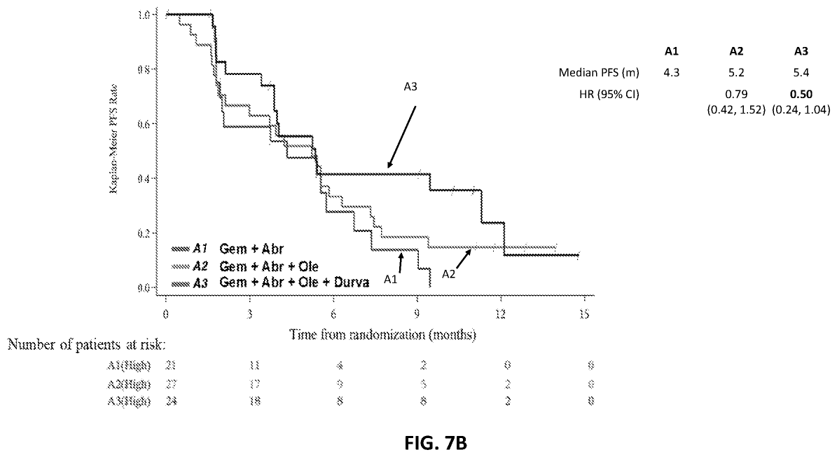

Confirmatory evidence showing that the addition of oleclumab+/−durvalumab to chemotherapy (gemcitabine/nab-paclitaxel) showed an increased overall response rate in the CD73 high sub-group and that the high CD73 expression sub-group was associated with shorter progression-free survival than the low CD73 sub-group regardless of the addition of oleclumab+/−durvalumab to chemotherapy (gemcitabine/nab-paclitaxel) is displayed in FIGS. 5A, 5B, 6A, 6B, 7A, 7B, and Table 4.

TABLE 4
Enrichment of response in CD73 high population at a minimum of 10 months follow up

| | CD73 Low | | | CD73 High (≥50% TC, 2+3+) | | |
|---|---|---|---|---|---|---|
| | Arm A1 | Arm A2 | Arm A3 | Arm A1 | Arm A2 | Arm A3 |
| | N = 7 | N = 10 | N = 8 | N = 19 | N = 25 | N = 22 |
| ORR, n (%) | 4 (57.1%) | 2 (20.0%) | 4 (50.0%) | 2 (10.5%) | 6 (24.0%) | 7 (31.8%) |
| [95% CI] | | | | [6.1%, 45.6%] | [14.9%, 53.5%] | [24.4%, 67.8%] |
| CR | 0 | 0 | 0 | 0 | 0 | 0 |
| PR | 4 (57.1%) | 2 (20.0%) | 4 (50.0%) | 2 (10.5%) | 6 (24.0%) | 7 (31.8%) |
| SD | 3 (42.9%) | 8 (80.0%) | 4 (50.0%) | 12 (63.2%) | 12 (48.0%) | 10 (45.5%) |
| Unconfirmed | 1 (14.3%) | 3 (30.0%) | 0 | 2 (10.5%) | 2 (8.0%) | 3 (13.6%) |
| PR | | | | | | |
| PD | 0 | 0 | 0 | 5 (26.3%) | 3 (12.0%) | 5 (22.7%) |
| NE | 0 | 0 | 0 | 0 | 4 (16.0%) | 0 |
| CR + PR (conf + | 5 (71.4%) | 5 (50.0%) | 4 (50.0%) | 4 (21.1%) | 8 (32.0%) | 10 (45.5%) |
| unconf) | | | | | | |
| DoR mo | NA | NA | NA | 6.4 (5.5, 7.2) | NA (2.2, NA) | 7.5 (1.9, 7.5) |

ORR = objective response rate
CR = complete response
PR = partial response
SD = stable disease
PD = progressive disease
NE = not evaluable
DoR = duration of response

The disclosure is not to be limited in scope by the specific aspects described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

What is claimed:

1. A method of treating a metastatic pancreatic ductal adenocarcinoma (PDAC) in a subject, the method comprising administering to the subject about 750 mg to about 3000 mg of an anti-CD73 antibody or antigen binding fragment thereof and chemotherapy, wherein a tumor sample obtained from the subject expresses CD73.

2. A method of inhibiting the growth of a PDAC tumor in a subject, the method comprising administering to the subject about 750 mg to about 3000 mg of an anti-CD73 antibody or antigen binding fragment thereof and chemotherapy, wherein a tumor sample obtained from the subject expresses CD73.

3. The method of claim 1 or claim 2, wherein the anti-CD73 antibody or antigen binding fragment thereof comprises oleclumab or antigen-binding fragment thereof.

4. The method of any one of claims 1-3, wherein the oleclumab or antigen-binding fragment thereof is administered at a dose of 750 mg.

5. The method of any one of claims 1-3, wherein the oleclumab or antigen-binding fragment thereof is administered at a dose of 1500 mg.

6. The method of any one of claims 1-3, wherein the oleclumab or antigen-binding fragment thereof is administered at a dose of 2250 mg.

7. The method of any one of claims 1-3, wherein the oleclumab or antigen-binding fragment thereof is administered at a dose of 3000 mg.

8. The method of any one of claims 1-7, wherein the dose of oleclumab or an antigen-binding fragment thereof is administered every two weeks for four doses and then every four weeks.

9. The method of any one of claims 1-8, wherein the chemotherapy comprises gemcitabine and nab-paclitaxel.

10. The method of claim 9, wherein the gemcitabine is administered at a dose of 1000 mg/m² and the nab-paclitaxel is administered at a dose of 125 mg/m².

11. The method of claim 9 or 10, wherein the chemotherapy is administered on days 1, 8, and 15 of a 28-day treatment cycle and then the cycle is repeated every 4 weeks.

12. The method of any one of claims 1-8, wherein the chemotherapy comprises mFOLFOX.

13. The method of claim 12, wherein the mFOLFOX comprises oxaliplatin administered at a dose of about 85 mg/m², leucovorin administered at a dose of about 400 mg/m², and 5-FU administered in a bolus of about 400 mg/m² followed by administration of a second dose of 5-FU at about 2400 mg/m².

14. The method of claim 12 or 13, wherein the chemotherapy is administered on days 1 and 15 of a 28-day treatment cycle and then the cycle is repeated every 4 weeks.

15. The method of any one of claims 1-11, wherein the oleclumab or antigen binding fragment thereof is administered at a dose of 750 mg every 2 weeks for four doses and then every 4 weeks in the treatment cycle, and wherein the chemotherapy comprises 1000 mg/m² gemcitabine and 125 mg/m² nab-paclitaxel and is administered on days 1, 8, and 15 of the 28-day treatment cycle and then the cycle is repeated every 4 weeks.

16. The method of any one of claims 1-11, wherein the oleclumab or antigen binding fragment thereof is administered at a dose of 1500 mg every 2 weeks for four doses and then every 4 weeks in the treatment cycle, and wherein the chemotherapy comprises 1000 mg/m² gemcitabine and 125 mg/m² nab-paclitaxel and is administered on days 1, 8, and 15 of the 28-day treatment cycle and then the cycle is repeated every 4 weeks.

17. The method of any one of claims 1-11, wherein the oleclumab or antigen binding fragment thereof is administered at a dose of 2250 mg every 2 weeks for four doses and then every 4 weeks in the treatment cycle, and wherein the chemotherapy comprises 1000 mg/m² gemcitabine and 125 mg/m² nab-paclitaxel and is administered on days 1, 8, and 15 of the 28-day treatment cycle and then the cycle is repeated every 4 weeks.

18. The method of any one of claims 1-11, wherein the oleclumab or antigen binding fragment thereof is administered at a dose of 3000 mg every 2 weeks for four doses and then every 4 weeks in the treatment cycle, and wherein the chemotherapy comprises 1000 mg/m² gemcitabine and 125 mg/m² nab-paclitaxel and is administered on days 1, 8, and 15 of the 28-day treatment cycle and then the cycle is repeated every 4 weeks.

19. The method of any one of claims 1-8 and 12-14, wherein the oleclumab or antigen binding fragment thereof is administered at a dose of 750 mg every 2 weeks for four doses and then every 4 weeks in the treatment cycle, and wherein the chemotherapy comprises mFOLFOX and is administered on days 1 and 15 of the 28-day treatment cycle and then the cycle is repeated every 4 weeks.

20. The method of any one of claims 1-11, wherein the oleclumab or antigen binding fragment thereof is administered at a dose of 1500 mg every 2 weeks for four doses and then every 4 weeks in the treatment cycle, and wherein the chemotherapy comprises mFOLFOX and is administered on days 1 and 15 of the 28-day treatment cycle and then the cycle is repeated every 4 weeks.

21. The method of any one of claims 1-11, wherein the oleclumab or antigen binding fragment thereof is administered at a dose of 2250 mg every 2 weeks for four doses and then every 4 weeks in the treatment cycle, and wherein the chemotherapy comprises mFOLFOX and is administered on days 1 and 15 of the 28-day treatment cycle and then the cycle is repeated every 4 weeks.

22. The method of any one of claims 1-11, wherein the oleclumab or antigen binding fragment thereof is administered at a dose of 3000 mg every 2 weeks for four doses and then every 4 weeks in the treatment cycle, and wherein the wherein the chemotherapy comprises mFOLFOX and is administered on days 1 and 15 of the 28-day treatment cycle and then the cycle is repeated every 4 weeks.

23. The method of any one of claims 1-22, wherein the oleclumab or antigen binding fragment and the chemotherapy are administered simultaneously or sequentially.

24. The method of any one of claims 1-23, further comprising administering to the subject about 1500 mg of an anti-PD-L1 antibody or antigen binding fragment thereof.

25. The method of claim 24, wherein the anti-PD-L1 antibody or antigen binding fragment thereof comprises durvalumab or antigen binding fragment thereof.

26. The method of claim 24 or 25, wherein the durvalumab or antigen binding fragment thereof is administered at a dose of 1500 mg.

27. The method of any one of claims 23-26, wherein the dose of durvalumab or antigen binding fragment thereof is administered every four weeks.

28. The method of any one of claims 23-27, wherein the oleclumab or antigen binding fragment thereof, the durvalumab or antigen binding fragment thereof, and the chemotherapy are administered simultaneously or sequentially

29. The method of any one of claims 1-28, wherein the administration is parenteral.

30. The method of claim 29, wherein the administration is intravenous.

31. The method of claim 29 or 30, wherein the administration is via intravenous infusion.

32. The method of any one of claims 1-31, wherein the subject is human.

33. The method of any one of claims 1-32, wherein the human subject is an adult ≥18 years of age with histologically or cytologically confirmed pancreatic adenocarcinoma.

34. The method of any one of claims 1-33, wherein the subject has previously untreated first-line metastatic PDAC (1L metastatic PDAC).

35. The method of any one of claims 1-33, wherein the subject has previously untreated second-line metastatic PDAC (2L metastatic PDAC).

36. The method of any one of claims 1-35, wherein the CD73 expression of a tumor sample obtained from the subject is evaluated by an immunohistochemistry (IHC) method.

37. The method of claim 36, wherein the IHC method is an automated IHC method.

38. The method of any one of claims 1-37, wherein a tumor sample obtained from the subject expresses high levels of CD73.

39. The method of any one of claims 1-38, wherein the IHC method comprises IHC scoring.

40. The method of claim 39, wherein the IHC scoring is defined by scoring the staining intensity of cells expressing CD73 within the tumor sample with the value 0, 1, 2, or 3.

41. The method of claim 39, wherein the IHC scoring is defined by scoring the staining intensity of cells expressing CD73 within the tumor sample with the value 1, 2, or 3.

42. The method of any one of claims 36-41, wherein the percentage of cells expressing CD73 at each value in the tumor sample is calculated.

43. The method of claim 42, wherein the tumor sample comprises cells having staining intensities of 1, 2, and 3.

44. The method of claim 40-43, wherein the tumor sample comprises at least about 50% to about 90% of cells having staining intensities of 1, 2, and 3.

45. The method of claim 44, wherein the tumor sample comprises at least about 50%, about 60%, about 70%, about 80%, or about 90% cells having staining intensities of 1, 2, and 3.

46. The method of any one of claims 40-43, wherein the tumor sample comprises cells having staining intensities of 2 and 3.

47. The method of claim 46, wherein the tumor sample comprises at least about 30% to about 70% of cells having staining intensities of 2 and 3.

48. The method of claim 47, wherein the tumor sample comprises at least about 30%, about 40%, about 50%, about 60%, or about 70% of cells having staining intensities of 2 and 3.

49. The method of any one of claims 1-48, wherein at least about 70% of the cells in a tumor sample obtained from the subject have a staining intensity score of at least 1.

50. The method of any one of claims 1-49, wherein at least about 50% of the cells in a tumor sample obtained from the subject have a staining intensity score of at least 2.

51. The method of any one of claims 1-50, wherein the anti-CD73 antibody or antigen binding fragment thereof comprises oleclumab which comprises a heavy chain variable domain and a light chain variable domain, wherein the heavy chain variable domain comprises CDR1, CDR2, and CDR3 sequences of SEQ ID NOs: 3-5, and wherein the light chain variable domain comprises CDR1, CDR2, and CDR3 sequences of SEQ ID NOs: 6-8.

52. The method of claim 51, wherein the oleclumab comprises a light chain variable domain comprising the amino acid sequence of SEQ ID NO: 1 and a heavy chain variable domain comprising the amino acid sequence of SEQ ID NO: 2.

53. The method of any one of claims 24-52, wherein the anti-PD-L1 antibody or antigen binding fragment thereof comprises durvalumab which comprises a heavy chain variable domain and a light chain variable domain, wherein the heavy chain variable domain comprises CDR1, CDR2, and CDR3 sequences of SEQ ID NOs: 11-13, and wherein the light chain variable domain comprises CDR1, CDR2, and CDR3 sequences of SEQ ID NOs: 14-16.

54. The method of claim 53, wherein the durvalumab comprises a light chain variable domain comprising the amino acid sequence of SEQ ID NO: 9 and a heavy chain variable domain comprising the amino acid sequence of SEQ ID NO: 10.

55. The method of any one of claims 1-52, wherein the anti-CD73 antibody or antigen binding fragment thereof is administered every 2 weeks for four doses and then every 4 weeks in a treatment cycle, and wherein the chemotherapy is administered on days 1, 8, and 15 of the 28-day cycle treatment cycle and then the cycle is repeated every 4 weeks.

56. The method of any one of claims 1-52, wherein the anti-CD73 antibody or antigen binding fragment thereof is administered every 2 weeks for four doses and then every 4 weeks in a treatment cycle, and wherein the chemotherapy is administered on days 1 and 15 of the 28-day cycle treatment cycle and then the cycle is repeated every 4 weeks.

57. The method of any one of claims 24-54, wherein anti-CD73 antibody or antigen binding fragment thereof is administered every 2 weeks for four doses and then every 4 weeks in a treatment cycle, and wherein the anti-PD-L1 antibody or antigen binding fragment thereof is administered every four weeks in the treatment cycle, wherein the chemotherapy is administered on days 1, 8, and 15 of the 28-day treatment cycle, and then the cycle is repeated every 4 weeks.

58. The method of any one of claims 36-56, wherein about 30% to about 70% of cells in a tumor sample obtained from the human subject have a staining intensity of at least 2.

59. The method of claim 58, wherein at least about 30%, about 35%, about 40%, about 45%, about 50%, about 55%, about 60%, about 65%, or about 70% of cells in a tumor sample obtained from the human subject have a staining intensity of at least 2.

60. A method of inhibiting the growth of a tumor in a human subject comprising administering (i) oleclumab or antigen binding fragment thereof, (ii) durvalumab or antigen binding fragment thereof, and (iii) chemotherapy to the subject, wherein at least about 50% to about 90% of cells in a tumor sample obtained from the human subject have a staining intensity of 1, 2, or 3, as determined by IHC;
and wherein the oleclumab or antigen binding fragment is administered to the subject at a dose of 750 mg every 2 weeks for four doses and then every 4 weeks in a treatment cycle; the durvalumab or antigen binding fragment thereof is administered to the subject at a dose of 1500 mg every four weeks in the treatment cycle; and the chemotherapy comprises 1000 mg/m² gemcitabine and 125 mg/m² nab-paclitaxel and is administered on days 1, 8, and 15 of the 28-day treatment cycle and then the cycle is repeated every 4 weeks.

61. A method of inhibiting the growth of a tumor in a human subject comprising administering (i) oleclumab or antigen binding fragment thereof, (ii) durvalumab or antigen binding fragment thereof, and (iii) chemotherapy to the subject, wherein at least about 50% to about 90% of cells in a tumor sample obtained from the human subject have a staining intensity of 1, 2, or 3, as determined by IHC;
and wherein the oleclumab or antigen binding fragment is administered to the subject at a dose of 1500 mg every 2 weeks for four doses and then every 4 weeks in a treatment cycle; the durvalumab or antigen binding fragment thereof is administered to the subject at a dose of 1500 mg every four weeks in the treatment cycle; and the chemotherapy comprises 1000 mg/m² gemcitabine and 125 mg/m² nab-paclitaxel and is administered on days 1, 8, and 15 of the 28-day cycle and then the cycle is repeated every 4 weeks.

62. A method of inhibiting the growth of a tumor in a human subject comprising administering (i) oleclumab or antigen binding fragment thereof, (ii) durvalumab or antigen binding fragment thereof, and (iii) chemotherapy to the subject, wherein at least about 50% to about 90% of cells in a tumor sample obtained from the human subject have a staining intensity of 1, 2, or 3, as determined by IHC;
and wherein the oleclumab or antigen binding fragment is administered to the subject at a dose of 2250 mg every 2 weeks for four doses and then every 4 weeks in a treatment cycle; the durvalumab or antigen binding fragment thereof is administered to the subject at a dose of 1500 mg every four weeks in the treatment cycle; and the chemotherapy comprises 1000 mg/m² gemcitabine and 125 mg/m² nab-paclitaxel and is administered on days 1, 8, and 15 of the 28-day cycle and then the cycle is repeated every 4 weeks.

63. A method of inhibiting the growth of a tumor in a human subject comprising administering (i) oleclumab or antigen binding fragment thereof, (ii) durvalumab or antigen binding fragment thereof, and (iii) chemotherapy to the subject, wherein at least about 50% to about 90% of cells in a tumor sample obtained from the human subject have a staining intensity of 1, 2, or 3, as determined by IHC;
and wherein the oleclumab or antigen binding fragment is administered to the subject at a dose of 3000 mg every 2 weeks for four doses and then every 4 weeks in a treatment cycle; the durvalumab or antigen binding fragment thereof is administered to the subject at a dose of 1500 mg every four weeks in the treatment cycle; and the chemotherapy comprises 1000 mg/m² gemcitabine and 125 mg/m² nab-paclitaxel and is administered on days 1, 8, and 15 of the 28-day cycle and then the cycle is repeated every 4 weeks.

64. A method of inhibiting the growth of a tumor in a human subject comprising administering (i) oleclumab or antigen binding fragment thereof, (ii) durvalumab or antigen binding fragment thereof, and (iii) chemotherapy to the subject, wherein at least about 50% to about 90% of cells in a tumor sample obtained from the human subject have a staining intensity of 1, 2, or 3, as determined by IHC;
and wherein the oleclumab or antigen binding fragment is administered to the subject at a dose of 750 mg every 2 weeks for four doses and then every 4 weeks in a treatment cycle; the durvalumab or antigen binding fragment thereof is administered to the subject at a dose of 1500 mg every four weeks in the treatment cycle; and the chemotherapy comprises mFOLFOX and is administered on days 1 and 15 of the 28-day treatment cycle and then the cycle is repeated every 4 weeks.

65. A method of inhibiting the growth of a tumor in a human subject comprising administering (i) oleclumab or antigen binding fragment thereof, (ii) durvalumab or antigen binding fragment thereof, and (iii) chemotherapy to the subject, wherein at least about 50% to about 90% of cells in a tumor sample obtained from the human subject have a staining intensity of 1, 2, or 3, as determined by IHC;
and wherein the oleclumab or antigen binding fragment is administered to the subject at a dose of 1500 mg every 2 weeks for four doses and then every 4 weeks in a treatment cycle; the durvalumab or antigen binding fragment thereof is administered to the subject at a dose of 1500 mg every four weeks in the treatment cycle; and the chemotherapy comprises mFOLFOX and is administered on days 1 and 15 of the 28-day treatment cycle and then the cycle is repeated every 4 weeks.

66. A method of inhibiting the growth of a tumor in a human subject comprising administering (i) oleclumab or antigen binding fragment thereof, (ii) durvalumab or antigen binding fragment thereof, and (iii) chemotherapy to the subject, wherein at least about 50% to about 90% of cells in a tumor sample obtained from the human subject have a staining intensity of 1, 2, or 3, as determined by IHC;
and wherein the oleclumab or antigen binding fragment is administered to the subject at a dose of 2250 mg every 2 weeks for four doses and then every 4 weeks in a treatment cycle; the durvalumab or antigen binding fragment thereof is administered to the subject at a dose of 1500 mg every four weeks in the treatment cycle; and the chemotherapy comprises mFOLFOX and is administered on days 1 and 15 of the 28-day treatment cycle and then the cycle is repeated every 4 weeks.

67. A method of inhibiting the growth of a tumor in a human subject comprising administering (i) oleclumab or antigen binding fragment thereof, (ii) durvalumab or antigen binding fragment thereof, and (iii) chemotherapy to the subject, wherein at least about 50% to about 90% of cells in a tumor sample obtained from the human subject have a staining intensity of 1, 2, or 3, as determined by IHC;
and wherein the oleclumab or antigen binding fragment is administered to the subject at a dose of 3000 mg every 2 weeks for four doses and then every 4 weeks in a treatment cycle; the durvalumab or antigen binding fragment thereof is administered to the subject at a dose of 1500 mg every four weeks in the treatment cycle; and the chemotherapy comprises mFOLFOX and is administered on days 1 and 15 of the 28-day treatment cycle and then the cycle is repeated every 4 weeks.

68. The method of any one of claims 60-67, wherein at least about 30% to about 70% of cells in a tumor sample obtained from the human subject have a staining intensity of at least 2.

69. The method of any one of claims 60-68, wherein at least about 30%, about 35%, about 40%, about 45%, about 50%, about 55%, about 60%, about 65%, or about 70% of cells in a tumor sample obtained from the human subject have a staining intensity of at least 2.

70. The method of any one of claims 60-69, wherein the tumor is a 1^{st} line metastatic pancreatic ductal adenocarcinoma.

71. The method of any one of claims 60-69, wherein the tumor is a 2^{nd} line metastatic pancreatic ductal adenocarcinoma.


SEQUENCE LISTING (16 sequences)

SEQ ID NO: 1 - protein, 110 aa, Artificial Sequence
  Oleclumab VL
QSVLTQPPSASGTPGQRVTISCSGSLSNIGRNPVNWYQQLPGTAPKLLIYLDNLRLSGVPDRFSGSKSGTSASLAISGLQSEDEADYYCATWDDSHPGWTFGGGTKLTVL

SEQ ID NO: 2 - protein, 117 aa, Artificial Sequence
  Oleclumab VH
EVQLLESGGGLVQPGGSLRLSCAASGFTFSSYAYSWVRQAPGKGLEWVSAISGSGGRTYYADSVKGRFTISRDNSKNTLYLQMNSLRAEDTAVYYCARLGYGRVDEWGRGTLVTVSS

SEQ ID NO: 3 - protein, 5 aa, Artificial Sequence
  Oleclumab VH CDR1
SYAYS

SEQ ID NO: 4 - protein, 17 aa, Artificial Sequence
  Oleclumab VH CDR2
AISGSGGRTYYADSVKG

SEQ ID NO: 5 - protein, 8 aa, Artificial Sequence
  Oleclumab VH CDR3
LGYGRVDE

SEQ ID NO: 6 - protein, 13 aa, Artificial Sequence
  Oleclumab VL CDR1
SGSLSNIGRNPVN

SEQ ID NO: 7 - protein, 7 aa, Artificial Sequence
  Oleclumab VL CDR2
LDNLRLS

SEQ ID NO: 8 - protein, 11 aa, Artificial Sequence
  Oleclumab VL CDR3
ATWDDSHPGWT

SEQ ID NO: 9 - protein, 108 aa, Artificial Sequence
  Durvalumab VL
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 10 - protein, 121 aa, Artificial Sequence
  Durvalumab VH
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 11 - protein, 10 aa, Artificial Sequence
  Durvalumab VH CDR1
GFTFSRYWMS

SEQ ID NO: 12 - protein, 17 aa, Artificial Sequence
  Durvalumab VH CDR2
NIKQDGSEKYYVDSVKG

SEQ ID NO: 13 - protein, 12 aa, Artificial Sequence
  Durvalumab VH CDR3
EGGWFGELAFDY

SEQ ID NO: 14 - protein, 12 aa, Artificial Sequence
  Durvalumab VL CDR1
RASQRVSSSYLA

SEQ ID NO: 15 - protein, 7 aa, Artificial Sequence
  Durvalumab VL CDR2
DASSRAT

SEQ ID NO: 16 - protein, 9 aa, Artificial Sequence
  Durvalumab VL CDR3
QQYGSLPWT